SEQUENCE LISTING (43 sequences)

SEQ ID NO: 1 - DNA, 1368 bp, Mycobacterium sp. JLS
  CDS (1)..(1368): JLS nidA coding sequence
  gene (1)..(1368)<s300> AY3301022004-06-24(1)..(1368)
atgaccaccgaaacaaccggaacagctgacgcgaccgatccctacctgcggcgcgcgttgcgggaggtagcggacgggctcaaggtcgggcgcttaccggcccgcgtcgtcagcgatcccgcgctacacacgatcgagatggagcggatcttcgggcgcgcctgggtgtttctcggacacgagtcggagttggccaagtccggcgacttcgtcgtgcggcacatcggggccgattcggtgatcgtttgccgggacaactccggccgcatccaggcgctgtccaattcttgtcgccaccgtggtgcgctcgtgtgccgggcggagatgggaaacaccgcgcacttccaatgcctgtaccacggctgggtgtacagcaacaccggagagctcgtcggcgtgccggcgatgacggaggcctatcccggcggcttcgacaagtcgcagtggggattacgtcacatcccccatgtcgactcgtacgccggattcatcttcggcagcgtcgatccgaaggcgccgagcctgaccgactacctcggcgacacgacgttctacctcgacctcattgcgaaaaaaacagcgggcggtctggaggtgataggggcaccgcatcgatgggtgacgtcagcgaactggaagacagccgccgacaattttgtcggcgactcctaccacaccctctttgctcaccgctcgatggtcgagctaggcatggcgcccggtgacccaaacttcgcgagcgcaccagcggaaatctcgctgcagaacggccacggcgtcggcgtactcggctttccgcccacgctcgccgattttcccgagtacgagggataccccgacgaagtcgtcgaccagatggcgacgtcctacccgtcgccggtacacaaggacctgatgcgacgctcatcctttattcacggcaccgtgttcccgaatttgtcgttcatcaacgtgaccctcgcgcaggaccacatgtcgccccctacccccttcatcacgttccgggtatggcatccgctctcccatgatcggatggagatcctctcctggttcctggtcgaacgcgatgctccggaatggttgcgcgatgcgtcccaggcgtcctacgtcaacaacttcggcccaggtggggttttcgaacaggacgacgccgaggcatggaaggccatcaccgaatctgtgcagggcccgttcgccggtgaaggcctgctgaactacgaaatgggcatggacttgactccgctcaccgactggccagggccgggagaggccctcccgagcgggtacgccgagcagaatcagcggcggttttgggggagatggctggaatacatgggtcagctccccgcattcggcgggcgtgcttga

SEQ ID NO: 2 - protein, 455 aa, Mycobacterium sp. JLS
MTTETTGTADATDPYLRRALREVADGLKVGRLPARVVSDPALHTIEMERIFGRAWVFLGHESELAKSGDFVVRHIGADSVIVCRDNSGRIQALSNSCRHRGALVCRAEMGNTAHFQCLYHGWVYSNTGELVGVPAMTEAYPGGFDKSQWGLRHIPHVDSYAGFIFGSVDPKAPSLTDYLGDTTFYLDLIAKKTAGGLEVIGAPHRWVTSANWKTAADNFVGDSYHTLFAHRSMVELGMAPGDPNFASAPAEISLQNGHGVGVLGFPPTLADFPEYEGYPDEVVDQMATSYPSPVHKDLMRRSSFIHGTVFPNLSFINVTLAQDHMSPPTPFITFRVWHPLSHDRMEILSWFLVERDAPEWLRDASQASYVNNFGPGGVFEQDDAEAWKAITESVQGPFAGEGLLNYEMGMDLTPLTDWPGPGEALPSGYAEQNQRRFWGRWLEYMGQLPAFGGRA

SEQ ID NO: 3 - DNA, 1368 bp, Mycobacterium sp. KMS
  CDS (1)..(1368): KMS nidA coding sequence <s300> AY330100 2004-06-24 (1)..(1368)
atgaccaccgaaacaaccggaacagctgacgcgaccgatccctacctgcggcgcgcgttgcgggaggtagcggacgggctcaaggtcgggcgcttaccggcccgcgtcgtcagcgatcccgcgctacacacgatcgagatggagcggatcttcgggcgcgcctgggtgtttctcggacacgagtcggagttggccaagtccggcgacttcgtcgtgcggcacatcggggccgattcggtgatcgtttgccgggacaactccggccgcatccaggcgctgtccaattcttgtcgccaccgtggtgcgctcgtgtgccgcgctgagatgggaaacaccgcgcacttccaatgcccgtaccacggctgggtgtacagcaacaccggagagctcgtcggcgtgccggcgatgagtgaggcctatcccggcggcttcgacaagtcgcagtggggattacgtcacatcccccatgtcgactcgtacgccggattcatcttcggcagcgtcgatccgaaggcgccgagcctgaccgactacctcggcgacacgacgttctacctcgacctcattgcgaagaagacagcgggcggtctggaggtgataggggcaccgcatcgatgggtgatgtcagcgaactggaagacagccgccgacaattttgtcggcgactcctaccacaccctctttgctcaccgctcgatggtcgagctaggcatggcgcccggtgacccaaacttcgcgagcgcaccagcggagatctcgctgcagaacggccacggcgtcggcgtactcggctttccgcccacgctcgccgattttcccgagtacgagggataccccgacgaagtcgtcgaccagatggcgacgtcctacccgtcgccggtacacaaggacctgatgcgacgctcatcctttattcacggcaccgtgttcccgaatttgtcgttcatcaacgtgaccctcgcgcaggaccacatgtcgccccctacccccttcatcacgttccgggtatggcatccgctctcccatgatcggatggagatcctctcctggttcctggtcgaacgcgatgctccggaatggttgcgcgatgcgtcccaggcgtcctacgtcaacaacttcggcccaggtggggttttcgaacaggacgacgccgaggcatggaaggccatcaccgaatctgtccagggcccgttcgccggtgaaggcctgctgaactacgaaatgggcatggacttgactccgctcaccgactggccagggccgggagaggccctcccgagcgggtacgccgagcagaatcagcggcggttttgggggaggtggctggaatacatgggtcagcctcccgcattcggcgggcgtgcttga

SEQ ID NO: 4 - protein, 455 aa, Mycobacterium sp. KMS
MTTETTGTADATDPYLRRALREVADGLKVGRLPARVVSDPALHTIEMERIFGRAWVFLGHESELAKSGDFVVRHIGADSVIVCRDNSGRIQALSNSCRHRGALVCRAEMGNTAHFQCPYHGWVYSNTGELVGVPAMSEAYPGGFDKSQWGLRHIPHVDSYAGFIFGSVDPKAPSLTDYLGDTTFYLDLIAKKTAGGLEVIGAPHRWVMSANWKTAADNFVGDSYHTLFAHRSMVELGMAPGDPNFASAPAEISLQNGHGVGVLGFPPTLADFPEYEGYPDEVVDQMATSYPSPVHKDLMRRSSFIHGTVFPNLSFINVTLAQDHMSPPTPFITFRVWHPLSHDRMEILSWFLVERDAPEWLRDASQASYVNNFGPGGVFEQDDAEAWKAITESVQGPFAGEGLLNYEMGMDLTPLTDWPGPGEALPSGYAEQNQRRFWGRWLEYMGQPPAFGGRA

SEQ ID NO: 5 - DNA, 1377 bp, Mycobacterium sp. MCS
  CDS (1)..(1377): MCS nidA coding sequence <s300> AY330102 2004-06-24 (1)..(1377)
atgaccaccgaaacaaccggacaccggcacacagctgacgcgaccgatccctacctgcggcgcgcgttgcgggaggtagcggacgggctcaaggtcgggcgcttaccggcccgcgtcgtcagcgatcccgcgctacacacgatcgagatggagcggatcttcgggcgcgcctgggtgtttctcggacacgagtcggagttggccaagtccggcgacttcgtcgtgcggcacatcggggctgattcggtgatcgtttgccgggacaactccggccgcatccaggcgctgtccaattcctgttcgtttgccgggacaactccggccgcatccaggcgctgtccaattcctgtcttccaatgcccgtaccacggctgggtgtacagcaacaccggagagctcgtcggcgtgccggcgatgagtgaggcctatcccggcggcttcgacaagtcgcagtggggattacgtcacatcccccatgtcgactcgtacgccggattcatcttcggcagcgtcgatccgaaggcgccgagcctgaccgactacctcggcgacacgacgttctacctcgacctcattgcgaagaagacagcgggcggtctggaggtgataggggcaccgcatcgatgggtgatgtcagcgaactggaagacagccgccgacaattttgtcggcgactcctaccacaccctctttgctcaccgctcgatggtcgagctaggcatggcgcccggtgacccaaacttcgcgagcgcaccagcggaaatctcgctgcagaacggccacggcgtcggcgtactcggctttccgcccacgctcgccgattttcccgagtacgagggataccccgacgaagtcgtcgaccagatggcgacgtcctacccgtcgccggtacacaaggacctgatgcgacgctcatcctttattcacggcaccgtgttcccgaatttgtcgttcatcaacgtgaccctcgcgcaggaccacatgtcgccccctacccccttcatcacgttccgggtatggcatccgctctcccatgatcggatggagatcctctcctggttcctggtcgaacgcgatgctccggaatggttgcgcgatgcgtcccaggcgtcctacgtcaacaacttcggcccaggtggggttttcgaacaggacgacgccgaggcatggaaggccatcaccgaatctgtccagggcccgttcgccggtgaaggcctgctgaactacgaaatgggcatggacttgactccgctcaccgactggccagggccgggagaggccctcccgagcgggtacgccgagcagaatcagcggcggttttgggggagatggctggaatacatgggtcagcctcccgcattcggcgggcgtgcttga

SEQ ID NO: 6 - protein, 458 aa, Mycobacterium sp. MCS
MTTETTGHRHTADATDPYLRRALREVADGLKVGRLPARVVSDPALHTIEMERIFGRAWVFLGHESELAKSGDFVVRHIGADSVIVCRDNSGRIQALSNSCSFAGTTPAASRRCPIPVFQCPYHGWVYSNTGELVGVPAMSEAYPGGFDKSQWGLRHIPHVDSYAGFIFGSVDPKAPSLTDYLGDTTFYLDLIAKKTAGGLEVIGAPHRWVMSANWKTAADNFVGDSYHTLFAHRSMVELGMAPGDPNFASAPAEISLQNGHGVGVLGFPPTLADFPEYEGYPDEVVDQMATSYPSPVHKDLMRRSSFIHGTVFPNLSFINVTLAQDHMSPPTPFITFRVWHPLSHDRMEILSWFLVERDAPEWLRDASQASYVNNFGPGGVFEQDDAEAWKAITESVQGPFAGEGLLNYEMGMDLTPLTDWPGPGEALPSGYAEQNQRRFWGRWLEYMGQPPAFGGRA

SEQ ID NO: 7 - DNA, 510 bp, Mycobacterium sp. JLS
  CDS (1)..(510): JLS nidB coding sequence <s300> AY330098 2004-06-24 (1)..(510)
atgaacgcggttgcggtcgatcgagatacccgtgaggctgtcgaggcgttcatgttccgggaggcggagctactcgatggggggcagttccgagaatggttgggtctgctcgaccccgacatccggtatgtggttccggtgcgcaccacccgtgaggattccgcgggttgggtgggcgcgatcgcgcattggaacgacgactacacgggcttggagatgcgggtccttcggggcgagacggatttctcgtgggccgagtcacctcggtcgcggacccggcacttcgtgtccaacattcgcacggttgccggaccggaggctgatgagttgaccgtgcgctcgaatctgttgttctttcgcagccgcggagatagcggccggtgggagttgctctcggccgaacgcgtcgacgtgttgcgccgcaccgacgattcgctgcggctcgctcggcgcgaggtgttactcgatcactcgacgttgcctatcgacaacctgtcggtagtcctgtag

SEQ ID NO: 8 - protein, 169 aa, Mycobacterium sp. JLS
MNAVAVDRDTREAVEAFMFREAELLDGGQFREWLGLLDPDIRYVVPVRTTREDSAGWVGAIAHWNDDYTGLEMRVLRGETDFSWAESPRSRTRHFVSNIRTVAGPEADELTVRSNLLFFRSRGDSGRWELLSAERVDVLRRTDDSLRLARREVLLDHSTLPIDNLSVVL

SEQ ID NO: 9 - DNA, 510 bp, Mycobacterium sp. KMS
  CDS (1)..(510): KMS nidB coding sequence <s300> AY330099 2004-06-24 (1)..(510)
atgaacgcggttgcggtcgatcgggatacccgtgaggctgtcgaggcgttcatgttccgggaggcggagctactcgatggggggcagttccgggaatggttgggtctgctcgaccccgacatccggtatgtggttccggtgcgcaccacccgtgaggattccgcgggttgggtgggtgcgatcgcgcattggaacgacgactacacgggcttggagatgcgggtccttcggggcgagacggatttctcgtgggccgagtcacctcggtcgcggacccggcacttcgtgtccaacattcgcacggttgccggaccggaggctgatgagttgaccgtgcgctcgaatctgttgttctttcgcagccgcggagatagcggccggtgggagttgctctcggccgaacgcgtcgacgtgttgcgccgcaccgacgattcgctgcggctcgctcggcgcgaggtgttactcgatcactcgacgttgcctatcgacaacctgtcggtagtcctgtag

SEQ ID NO: 10 - protein, 169 aa, Mycobacterium sp. KMS; identical to SEQ ID NO: 8

SEQ ID NO: 11 - DNA, 510 bp, Mycobacterium sp. MCS
  CDS (1)..(510): MCS nidB coding sequence <s300> AY330101 2004-06-24 (1)..(510)
atgaacgcggttgcggtcgatcgagatacccgtgaggctgtcgaggcgttcatgttccgggaggcggagctactcgatggggggcagttccgagaatggttgggtctgctcgaccccgacatccggtatgtggttccggtgcgcaccacccgtgaggattccgcgggttgggtgggcgcgatcgcgcactggaacgacgactacacgggcttggagatgcgggtcctccggggcgagacggatttctcgtgggccgagtcacctcggtcgcggacccggcacttcgtgtccaacatccgcacggctgccggaccggaggctgatgagttgaccgtgcgctcaaatctcttgttcttccgcagccgcggagacagcggccggtgggagttgctctcggccgaacgcgtcgacgtgttgcgccgcaccgacgattcgctgcggctcgctcggcgcgaggtgttactcgatcactcgacgttgcctatcgacaacctgtcggtagtcctgtag

SEQ ID NO: 12 - protein, 169 aa, Mycobacterium sp. MCS
MNAVAVDRDTREAVEAFMFREAELLDGGQFREWLGLLDPDIRYVVPVRTTREDSAGWVGAIAHWNDDYTGLEMRVLRGETDFSWAESPRSRTRHFVSNIRTAAGPEADELTVRSNLLFFRSRGDSGRWELLSAERVDVLRRTDDSLRLARREVLLDHSTLPIDNLSVVL

SEQ ID NO: 13 - DNA, 295 bp, Mycobacterium sp. JLS
  promoter (1)..(295): JLS nidB promoter sequence
gcgcacattgcaagggccgccgccgctgtcaagccgctggcaaggcgacgacgaccgagcgatgcctctggtcgtcgtctatgggttcgctggctgtcgacgacgtgtgccgtggggctggttctgggagcgccgtggggcttgctgtgggagccgttttggtcatggtccattcggagatgttgtcttgacggcccggtgagacgtgacttacattctctacagcggaggacgcatctgttcagcagaatagagagtgagtgttgatggtctggtcgaggagttcggtgtgatg

SEQ ID NO: 14 - DNA, 295 bp, Mycobacterium sp. KMS
  promoter (1)..(295): KMS nidB promoter sequence
gcgcacattgcaagggccgccgccgctgtcaagccgctggcaaggcgacgacgaccgagcgatgcctctggtcgtcgtctatgggttcgctggctgtcgacgacgtgtgccgtggggctggttctgggagcgccgtggggcttgctgtgggagccgttttggtcatggtccattcggagatgttgtcttgacggcccggtgagacgtgacttacattctctacagcggaggacgcatctgttcagcagaatatggagtcagtgttgatggtctggtcgaggagttcggtgtgatg

SEQ ID NO: 15 - DNA, 295 bp, Mycobacterium sp. MCS; identical to SEQ ID NO: 14
  promoter (1)..(295): MCS nidB Promoter Sequence

SEQ ID NO: 16 - DNA, 295 bp, Mycobacterium flavescens
  promoter (1)..(295): Flav nidB promotor sequence
  promoter (1)..(295): Flav nidB promoter sequence
gcgcacattgcaagggccgccgccgctgtcaagccgctggcaaggcgacgacgaccgagcgatgcctctggtcgtcgtctatgggttcgctggctgtcgacgacgtgtgccgtggggctggttctgggagcgccgtggggcttgctgtgggagccgttttggtcatggtccattcggagatgttgtctcgacggcccggtgagacgtagcttacattctctacagcggaggacgcatctgttcagcagaatgtcgagtcagtgttgatggtctggtcgaggagttcggtgtgatg

SEQ ID NO: 17 - DNA, 295 bp, Mycobacterium vanbaalenii; identical to SEQ ID NO: 13
  promoter (1)..(295): Pyr-1 nidB promotor sequence
  promoter (1)..(295): Pyr-1 nidB promoter sequence

SEQ ID NO: 18 - DNA, 421 bp, Mycobacterium sp. JLS
  promoter (1)..(421): JLS 16S promoter sequence
cctgaagagtctgggcgccgagatcgagcgcgtggtgtagtcgccgacggtctgacgccgcggaaatcgcggttgtcacgcctctgaccaggcgattttggcttcggcgcaacctgtgcgtaagctaacggacataccgaaggcccccggccgaaaggcccgggaggaacctggttgacttcctcgaaacaatgcagtacagtatcggggttgcctgctcagcgggtgtgttgtttgagaacacaatagtgtgtttggtggtttttgtttgttgtttttgttttggccgcgcctctttttcccgtttaggggtgtggttgtttttggatgccagtttttggtgtcttgttgtttgatcagatttttctgaatggcctttttgggttttgtttggagagtttgattctggctcaggacgaac

SEQ ID NO: 19 - DNA, 421 bp, Mycobacterium sp. KMS
  promoter (1)..(421): KMS 16S promoter sequence
cctgaagagtctgggcgccgagatcgagcgcgtggtgtagtcgccgacggtctgacgccgcggaaatcgcggttgtcacgcctctgaccaggcgattttggcttcggcgcaacctgtgcgtaagctaacggacataccgaaggcccccggccgaaaggcccgggaggaacctggtcgacttcctcgaaacaatgcagtacagtatcggggttgcctgctcagcgggtgtgttgtttgagaactcaatagtgtgtttggtggtttttgtttgttgtttttgttttggccgcgcctctttttcccgtttaggggtgtggttgtttttggatgccagttcttggtgtcttgttgtttgatcagatttttctgaatggcctttttgggttttgtttggagagtttgattctggcttaggacgaac

SEQ ID NO: 20 - DNA, 421 bp, Mycobacterium sp. MCS
  promoter (1)..(421): MCS 16S promoter sequence
cctgaagagtctgggcgccgagatcgagcgcgtggtgtagccgccgacggtctgacgccgcggaaatcgcggttgtcacgcctctgaccaggcgattttggcttcggcgcaacctgtgcgtaagctaacggacataccgaaggcccccggccgaaaggcccgggaggaacctggttgacttcctcgaaacaatgcagtacagtatcggggttgcctgctcagcgggtgtgttgtttgagaactcaatagtgtgtttggtggtttttgtttgttgtttttgttttggccgcgcctctttttcccgtttaggggtgtggttgtttttggatgccagtttttggtgtcttgttgtttgatcagatttttctgaatggcctttttgggttttgtttggagagtttgattctggctcaggacgaac

SEQ ID NO: 21 - DNA, 420 bp, Mycobacterium flavescens
  promoter (1)..(420): Flav 16S promotor sequence
  promoter (1)..(420): Flav 16S promoter sequence
tcttgccgcactgggcgcggagatcgagcgcgtcacgtaacgtcgatgggtccgaaaagcccttctacgctggcgatttggaaccttgcgcgaagaccgcgtagtgttaccgacatcagcagcgagcccccgggggtccggaaggaaatccggggcagcggcttgaccgcccgccagggctgttatagactggcggggttgccttaaagcaggctctgttgtttgagaactcaatagtgtgtttggtggtttttgtttgttgtttttttgttgtgtcctcttttcccgtttgggggcatggcttttttgatgccagtttttggtgtcttgtttgatgatcggatttctctgattgtgaattcacctgtctgtggatgggttgtttttgtttggagagtttgattctggctcaggacgaac

SEQ ID NO: 22 - DNA, 428 bp, Mycobacterium vanbaalenii
  promoter (1)..(428): Pyr-1 16S promotor sequence
  promoter (1)..(428): Pyr-1 16S promoter sequence
tcttgtcagcctgggcgcggagatcgagcgggtaaactagtcctagtcccgcagtgagagacgtgcaagcctctgaccagcggatttgacttcgaaagaacgcgagttctaatcttatgaagtcgccgcggaaacgcggaacacagaagtttgactcgccggcgaagatagtttaagctggcagggttgccctgaaaaggggtaaccaataaaatctggtctgttgtttgagaactcaatagtgtgtttggtggtttttgtttgttgttttttggcctgcctctttttcccgtttaggggtgggttgtttttgatgccagttttggtgtcttgtttgatgatcggatttctctgattgtgaattctgcctgcgccctttgggtgtgggggtctttgtttggagagtttgattctggctcaggacgaac

SEQ ID NO: 23 - DNA, 1453 bp, Mycobacterium sp. JLS
  misc_feature (1)..(1453): Mycobacterium sp. JLS 16S ribosomal RNA gene, partial sequence. <s300> AF387804 2001-07-05 (1)..(1453)
gctggcggcgtgcttaacacatgcaagtcgaacggaaaggcccttcggggtactcgagtggcgaacgggtgagtaacacgtgggtgatctgccctgcactttgggataagcctgggaaactgggtctaataccgaatacaccctgctggtcgcatggcctggtgggggaaagcttttgcggtgtgggatgggcccgcggcctatcagcttgttggtgaggttacggctcaccaaggcgacgacgggtagccggcctgagagggtgaccggccacactgggactgagatacggcccagactcctacgggaggcagcagtggggaatattgcacaatgggcgcaagcctgatgcagcgacgccgcgtgggggatgacggccttcgggttgtaaacctctttcagtagggacgaagcgcaagtgacggtacctacagaagaaggaccggccaactacgtgccagcagccgcggtaatacgtagggtccgagcgttgtccggaattactgggcgtaaagagctcgtaggtggtttgtcgcgttgttcgtgaaaaccgggggcttaaccctcggcgtgcgggcgatacgggcagacttgagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgctggtagagatatcagttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggta

SEQ ID NO: 24 - DNA, 1454 bp, Mycobacterium sp. KMS
  misc_feature (1)..(1454): Mycobacterium sp. KMS 16S ribosomal RNA gene, partial sequence. <s300> AY083217 2002-03-23 (1)..(1454)
gctggcggcgtgcttaacacatgcaagtcgaacggaaaggcccttcggggtgctcgagtggcgaacgggtgagtaacacgtgggtgatctgccctgcactttgggataagcctgggaaactgggtctaataccgaatacaccctgctggtcgcatggcctggtgggggaaagcttttgcggtgtgggatgggcccgcggcctatcagcttgttggtgaggttacggctcaccaaggcgacgacgggtagccggcctgagagggtgaccggccacactgggactgagatacggcccagactcctacgggaggcagcagtggggaatattgcacaatgggcgcaagcctgatgcagcgacgccgcgtgggggatgacggccttcgggttgtaaacctctttcagtagggacgaagcgcaagtgacggtacctacagaagaaggaccggccaactacgtgccagcagccgcggtaatacgtagggtccgagcgttgtccggaattactgggcgtaaagagctcgtaggtggtttgtcgcgttgttcgtgaaaaccgggggcttaaccctcggcgtgcgggcgatacgggcagacttgagtattgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgctggtagagatatcagttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtaa

SEQ ID NO: 25 - DNA, 1394 bp, Mycobacterium sp. MCS
  misc_feature (1)..(1394): Mycobacterium sp. MCS 16S ribosomal RNA gene, partial sequence. <s300> AF387803 2001-07-05 (1)..(1394)
gctggcggcgtgcttaacacatgcaagtcgaacggaaaggcccttcggggtgctcgagtggcgaacgggtgagtaacacgtgggtgatctgccctgcactttgggataagcctgggaaactgggtctaataccgaatacaccctgctggtcgcatggcctggtgggggaaagcttttgcggtgtgggatgggcccgcggcctatcagcttgttggtgaggttacggctcaccaaggcgacgacgggtagccggcctgagagggtgaccggccacactgggactgagatacggcccagactcctacgggaggcagcagtggggaatattgcacaatgggcgcaagcctgatgcagcgacgccgcgtgggggatgacggccttcgggttgtaaacctctttcagtagggacgaagcgcaagtgacggtacctacagaagaaggaccggccaactacgtgccagcagccgcggtaatacgtagggtccgagcgttgtccggaattactgggcgtaaagagctcgtaggtggtttgtcgcgttgttcgtgaaaaccgggggcttaaccctcggcgtgcgggcgatacgggcagacttgagtattgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgctggtagagatatcagttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccc

SEQ ID NO: 26 - DNA, 21 bp, Mycobacterium vanbaalenii
  misc_binding (1)..(21): Primer for nidA 1st half of subunit
atgaccaccgaaacaaccgga

SEQ ID NO: 27 - DNA, 21 bp, Mycobacterium vanbaalenii
  misc_binding (1)..(21): Primer for nidA Reverse
tcaagcacgcccgccgaatgc

SEQ ID NO: 28 - DNA, 21 bp, Mycobacterium vanbaalenii
  misc_binding (1)..(21): Primer nidA Forward 642-633
gccgccgacaattttgtcggc

SEQ ID NO: 29 - DNA, 21 bp, Mycobacterium vanbaalenii
  misc_binding (1)..(21): Primer nidA Reverse 735-714
gaagtttgggtcaccgggcgc

SEQ ID NO: 30 - DNA, 21 bp, Mycobacterium vanbaalenii
  misc_binding (1)..(21): Primer nidB Forward
atgaacgcggttgcggtcgat

SEQ ID NO: 31 - DNA, 21 bp, Mycobacterium vanbaalenii
  misc_binding (1)..(21): Primer nidB Reverse
ctacaggactaccgacaggtt

SEQ ID NO: 32 - DNA, 21 bp, Mycobacterium vanbaalenii
  misc_binding (1)..(21): Primer nidB Forward 207-228
ggtttggagatgcgggtcctt

SEQ ID NO: 33 - DNA, 21 bp, Mycobacterium vanbaalenii
  misc_binding (1)..(21): Primer nidB Reverse 297-276
aatgttagacacgaagtgccg

SEQ ID NO: 34 - DNA, 21 bp, Mycobacterium vanbaalenii
  misc_binding (1)..(21): Primer nidB-nidA Forward 270-290
gcggacccggcacttcgtgtc

SEQ ID NO: 35 - DNA, 21 bp, Mycobacterium vanbaalenii
  misc_binding (1)..(21): Primer nidB-nidA Reverse 171-151
aaacacccaggcgcgcccgaa

SEQ ID NO: 36 - DNA, 21 bp, Mycobacterium vanbaalenii
  misc_binding (1)..(21): Forward Primer for Mycobacterium 16S ribosomal DNA
tggagagtttgatcctggctc

SEQ ID NO: 37 - DNA, 20 bp, Mycobacterium vanbaalenii
  misc_binding (1)..(20): Reverse Primer Mycobacterium 16S Ribosomal DNA
cggctgctggcacgtagttg

SEQ ID NO: 38 - DNA, 20 bp, Mycobacterium vanbaalenii
  misc_binding (1)..(20)
  misc_binding (1)..(20): Forward Primer for Mycobacterium 16S ribosomal DNA
tcgggttgtaaacctctttc

SEQ ID NO: 39 - DNA, 20 bp, Mycobacterium vanbaalenii
  misc_binding (1)..(20): Reverse Primer for Mycobacterium 16S ribosomal DNA
gacgacagccatgcaccacc

SEQ ID NO: 40 - DNA, 20 bp, Mycobacterium vanbaalenii
  misc_binding (1)..(20): Forward Primer for Mycobacterium 16S ribosomal DNA
gggtttgacatgcacaggac

SEQ ID NO: 41 - DNA, 21 bp, Mycobacterium vanbaalenii
  misc_binding (1)..(21): Reverse Primer for Mycobacterium 16S ribosomal DNA
taccttgttacgacttcgtcc

SEQ ID NO: 42 - DNA, 21 bp, Mycobacterium sp. JLS
  misc_feature (1)..(21): nidBpro-left is located from -124 to -104 bases upstream (5′) to the nidB start ATG.
tccattcggagatgttgtctt

SEQ ID NO: 43 - DNA, 21 bp, Mycobacterium sp. JLS
  misc_feature (1)..(21): nidBpro-right is located from -33 to -13 bases upstream (5′) to the nidB start ATG
ctcgaccagaccatcaacact

(19) United States
(12) Patent Application Publication
(10) Pub. No.: US 2007/0134683 A2
(43) Pub. Date: Jun. 14, 2007

(54) PROBES AND METHODS FOR IDENTIFYING POLYCYCLIC AROMATIC HYDROCARBON-DEGRADING MYCOBACTERIA

(76) Inventors: Charles D. Miller, North Logan, UT (US); Anne J. Anderson, Providence, UT (US); Ronald C. Sims, Logan, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/446,411

(22) Filed: Jun. 2, 2006

Related U.S. Application Data
(63) Continuation of application No. 60/687,567, filed on Jun. 3, 2005.
Application No. 60/687,567 is a continuation of application No. 60/693,452, filed on Jun. 23, 2005.

(51) Int. Cl. C12Q 1/68; C07H 21/04

(52) U.S. Cl. 435/6; 536/24.1

(57) ABSTRACT

A method for determining whether a microorganism is capable of biodegrading a contaminant can include the following: providing a first set of DNA molecules consisting of fragments of genomic DNA of a contaminant-degrading mycobacterium; contacting under hybridizing conditions, the first set of DNA molecules with a second set of DNA molecules consisting of genomic DNA of a microorganism, wherein it is not known whether or not the microorganism biodegrades a contaminant; and detecting hybridization between the first set of DNA molecules and the second set of DNA molecules, wherein the hybridization between the first and second sets is an indication that the microorganism is capable of biodegrading a contaminant.

### CROSS-REFERENCE TO RELATED APPLICATIONS

This United States Patent Application claims benefit of U.S. Provisional Patent Application Ser. No. 60/687,567, entitled “IDENTIFYING AND PROPAGATING POLYCYCLIC AROMATIC HYDROCARBON-DEGRADING MYCOBACTERIA,” filed on Jun. 3, 2005, with Charles D. Miller, Anne J. Anderson, and Ronald C. Sims as inventors, and also claims benefit of U.S. Provisional Patent Application Ser. No. 60/693,452, entitled “PROBES AND METHODS FOR IDENTIFYING POLYCYCLIC AROMATIC HYDROCARBON-DEGRADING MYCOBACTERIA,” filed Jun. 23, 2005, with Charles D. Miller, Anne J. Anderson, and Ronald C. Sims as inventors, which are incorporated herein by reference. This United States Patent Application cross-references United States Patent Application having Attorney Docket No. 14185.7.3.2, entitled “MYCOBACTERIA COMPOSITIONS AND METHODS OF USE IN BIOREMEDIATION,” filed concurrently herewith, which is incorporated herein by reference.

The U.S. Government has a paid-up license in this invention and the right in limited circumstances to require the patent owner to license others on reasonable terms as provided for by the terms of Grant No. A08379 awarded by the National Science Foundation.

### BACKGROUND OF THE INVENTION

1. The Field of the Invention

The present invention relates to DNA sequences for use as probes and/or primers. More particularly, the present invention relates to probes and/or PCR primers for use in methods of identifying contaminant-degrading mycobacteria in samples.

2. The Related Technology

Many industries use and/or generate toxic chemicals in systems, equipment, and processes during the production of the vast array of commercial products on the market even though the products themselves may or may not present toxic characteristics. As a consequence the soil and environment near or downstream from industrial sites often becomes contaminated. While various remediation techniques have been developed to decontaminate soil, various complex organic compounds are difficult to remove or break down. Examples of noxious soil contaminates include the organic compounds known as polycyclic aromatic hydrocarbons (“PAH”), polychlorinated phenols (“PCP”), and methyl tertiary butyl ether (“MTBE”), which are commonly present in soil around industrial sites and have toxic, mutagenic, and carcinogenic properties

Various types of soil remediation techniques have been developed in order to remove PAHs, PCPs, MTBEs, and other contaminates from the areas surrounding abandoned industrial sites. Bioremediation of soils has been shown to be a promising technique when microorganisms were determined to be capable of naturally degrading the contaminating contaminant chemicals. However, bioremediation may not be a suitable technique when contaminant-degrading microorganisms are not available for degrading a particular chemical or class of chemicals (e.g., PAH, PCP, MTBE) present in a site needing decontamination.

Therefore, it would be advantageous to identify microorganisms that are capable of degrading various soil contaminates, such as low molecular weight and/or high molecular weight PAHs, PCPs, MTBEs, and the like. Additionally, it would be beneficial to be capable of assessing the presence of contaminant-degrading microorganism in contaminated soils so that the microorganisms can be used for bioremediation.

### BRIEF SUMMARY OF THE INVENTION

Generally, the foregoing deficiencies in the art can be solved by embodiments of the present invention which can be employed to identify microorganisms that are capable of degrading various soil contaminates such as low molecular weight and/or high molecular weight PAHs. Additionally, embodiments of the present invention can be employed to assess whether or not a PAH-degrading microorganism is present in PAH-contaminated soil so that the microorganisms can be used for bioremediation.

One embodiment of the present invention is a method for determining whether a microorganism is a PAH-degrading mycobacterium. Such a method includes: providing a first set of DNA molecules consisting of fragments of genomic DNA of at least one mycobacterium species capable of biodegrading a PAH; contacting, under hybridizing conditions, the first set of DNA molecules with a second set of DNA molecules consisting of genomic DNA of an unknown mycobacterium species isolated from a sample; and detecting hybridization between the first set of DNA molecules and the second set of DNA molecules, wherein the hybridization between the first and second sets is an indication that the unknown mycobacteria species is PAH-degrading mycobacteria.

One embodiment of the present invention is a method of identifying the presence of PAH-degrading mycobacteria having nidB-nidA dioxygenase genes in a soil sample. Such a method includes: providing at least one primer capable of hybridizing with a nid dioxygenase nucleotide sequence; hybridizing the at least one primer with the nid dioxygenase nucleotide sequence; producing a polymerase chain reaction (“PCR”) product; and determining whether the PCR product indicates the presence of a PAH-degrading mycobacterium.

One embodiment of the present invention is a method of using genomic DNA of a known PAH-degrading mycobacterium to determine whether a sample, such as a soil sample or extract thereof, contains PAH-degrading mycobacteria. Such a method includes producing a nucleotide sequence which consists of a nidB and/or nidA DNA sequence of at least one of SEQ ID NO: 1, SEQ ID NO: 3, SEQ ID NO: 5, SEQ ID NO: 7, SEQ ID NO: 9, SEQ ID NO: 11, or complements thereof.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIGS. 1A-1D are flow diagrams illustrating embodiments of methods for preparing a sample and methods for identifying PAH-degrading mycobacteria in soil;

FIGS. 2A-2B are gel electrophoresis of PAH-degrading mycobacterial enzymes;

FIG. 3 is a table illustrating characteristics of PAH-degrading mycobacteria;

FIG. 4 is a graph illustrating mineralization of pyrene by mycobacteria;

FIG. 5 is a table illustrating embodiments of PCR primers(nidA forward (SEQ ID NO: 26): ATGACCACCGAAACAACCGGA; nidA 735-714 reverse (SEQ ID NO: 33): AATGTTAGACACGAAGTGCCG; nidA reverse (SEQ ID NO: 27): TCAAGCACGCCCGCCGAATGC; nidB forward (SEQ ID NO: 30): ATGAACGCGGTTGCGGTCGAT; nidB reverse (SEQ ID NO: 31): CTACAGGACTACCGACAGGTT; nidB 270-290 forward (SEQ ID NO: 34): GCGGACCCGGCACTTCGTGTC; nidA 171-151 (SEQ ID NO: 35): AAACACCCAGGCGCGCCCGAA);

FIGS. 6A-6C are gel electrophoresis of DNA extracted from soil;

FIG. 7 is a table comparing nid DNA of PAH-degrading mycobacteria;

FIG. 8 is a gel electrophoresis of PCR products using primers from a nidB promoter region;

FIG. 9 is a graph illustrating the ability of mycobacteria to form a biofilm;

FIG. 10 is a graph illustrating the ability of mycobacteria to have planktonic growth;

FIG. 11 is a graph illustrating mycobacterial colony forming units on roots when plants are grown in a microbially-contaminated soil mix;

FIGS. 12A-12B are photographs illustrating mycobacterial colonies on roots growing on plate medium from roots of seedlings grown from inoculated barley seeds;

FIGS. 13A-13D are graphs illustrating mycobacterial colony forming units on root sections along the length of the root;

FIG. 14 is a graph illustrating pyrene mineralization in microcosms containing barley with and without root colonization with a mycobacterium or with just microbial amendment of the growth medium;

FIG. 15 is a table showing the mass balance for recovery of label from radioactive pyrene from the microcosms described in FIG. 7;

FIG. 16 is a graph illustrating MTBE mineralization;

FIG. 17 is a graph illustrating TBA mineralization; and

FIG. 18 is a graph illustrating MTBE and TBA mineralization.

### DETAILED DESCRIPTION OF THE PREFERRED EMBODIMENTS

Generally, embodiments of the present invention are related to DNA sequences of contaminant-degrading mycobacteria and methods of using such DNA sequences to determine whether a sample has an unknown microorganism capable of biodegrading a contaminant, such as a PAH. Also, a microorganism identified as capable of degrading a PAH may also be capable of degrading other contaminants, such as PCPs, MTBEs, and like contaminants. Also, the DNA nucleotide sequences provided in the Sequence Listing below can be used in order to determine whether or not a sample, such as a soil sample or DNA extract thereof, contains genes that encode for nid dioxygenase.

I. Introduction

Nid dioxygenase genes, especially those having the nidB-nidA sequence motifs, have been shown by the inventors to be present in microorganisms that can biodegrade PAHs. Assays that can identify the presence of nid dioxygenase genes in various types of samples can be valuable for finding new PAH-biodegrading microorganisms and determining whether or not PAH-contaminated soils contain such microorganisms. Additionally, such assays that can identify the presence of nid dioxygenase genes in various types of samples can be valuable for finding microorganisms capable of degrading other types of contaminants, such as PCPs, MTBEs, and like contaminants. Thus, new methods and assays are presented herein that do not rely on time consuming and tedious methods that require culturing microorganisms on contaminant-contaminated mediums, which can take days and are fraught with uncertainty.

One embodiment of the present invention is a method for determining whether a microorganism is capable of biodegrading a contaminant, such as PAH, by assaying for the presence of nid dioxygenase DNA sequences in the genome. Such a method is performed when there is not any indication the microorganism has the ability to degrade PAHs. A microorganism can be shown to be capable of degrading PAHs by having the ability to grow in a medium that includes the presence of PAHs. In part, this is because PAHs are known to be toxins to most living organisms, and the ability to grow and replicate in a PAH-contaminated environment indicates PAH-biodegradability. While PAH-degrading mycobacteria are described herein, it should be realized that PAH-degrading mycobacteria can also degrade other contaminants, such as PCPs, MTBEs, and like contaminants.

Previously, the inventors showed that PAH-biodegrading microorganisms can be found in PAH-contaminated soils. The microorganisms, such as Mycobacterium JLS (“JLS”), Mycobacterium KMS (“KMS”), and Mycobacterium MCS (“MCS”) isolates, where shown to be capable of biodegrading PAHs by being cultured on a medium in the presence of a PAH such as pyrene. Additionally, the inventors showed further PAH-biodegradation capabilities by these isolates utilizing phenathrene and benzo[a]pyrene. Furthermore, the contaminant-degrading mycobacteria can include selected gene sequences that identify the capability of degrading selected contaminants, such as selected gene sequences that identify the capability of degrading PCPs, MTBEs, or other similar selected contaminants.

Additionally, the inventors showed that the PAH-biodegrading microorganisms can be identified by analysis of their fatty acid content and 16S ribosomal genes. As such, MIDI (Newark, Del.) performed analysis of the fatty acid content as described in the incorporated references. Also, the 16S ribosomal genes were assayed by PCR analysis with primers identified by SEQ ID NO: 36, SEQ ID NO: 37, SEQ ID NO: 38, SEQ ID NO: 39, SEQ ID NO: 40, and SEQ ID NO: 41. The fatty acid content and 16S ribosomal gene analysis provided a phylogenic indication that the microorganisms were mycobacterium. A phylogenic analysis indicated JLS, KMS, and MCS to be related to other mycobacterium that have PAH-biodegrading capabilities.

The foregoing experimental techniques illustrated that PAH-biodegrading microorganisms can be isolated from soils by being cultured on PAH-contaminated mediums and characterized by assessing the 16S ribosomal gene relation to other species. While the foregoing experimental techniques can be employed to find and identify PAH-biodegrading microorganisms, the present invention provides an improvement for identifying the presence of contaminant-degrading mycobacterium by isolating DNA directly from a soil sample and amplifying nid dioxygenase genes.

II. Method of Preparing Samples

In accordance with the present invention, samples can be prepared in order to determine whether or not they include contaminant-degrading mycobacteria. Such samples can be prepared directly from soil that is in or around sites known to be contaminated with contaminants. The methods of sample preparation can be performed before subsequent genetic analysis, or prepared by an external source and then delivered to a facility for the genetic analysis as described below. While the following methods are described with respect to PAH-degrading mycobacteria, the contaminant can be any contaminant, especially PCPs, MTBEs, TBAs, and other similar contaminants.

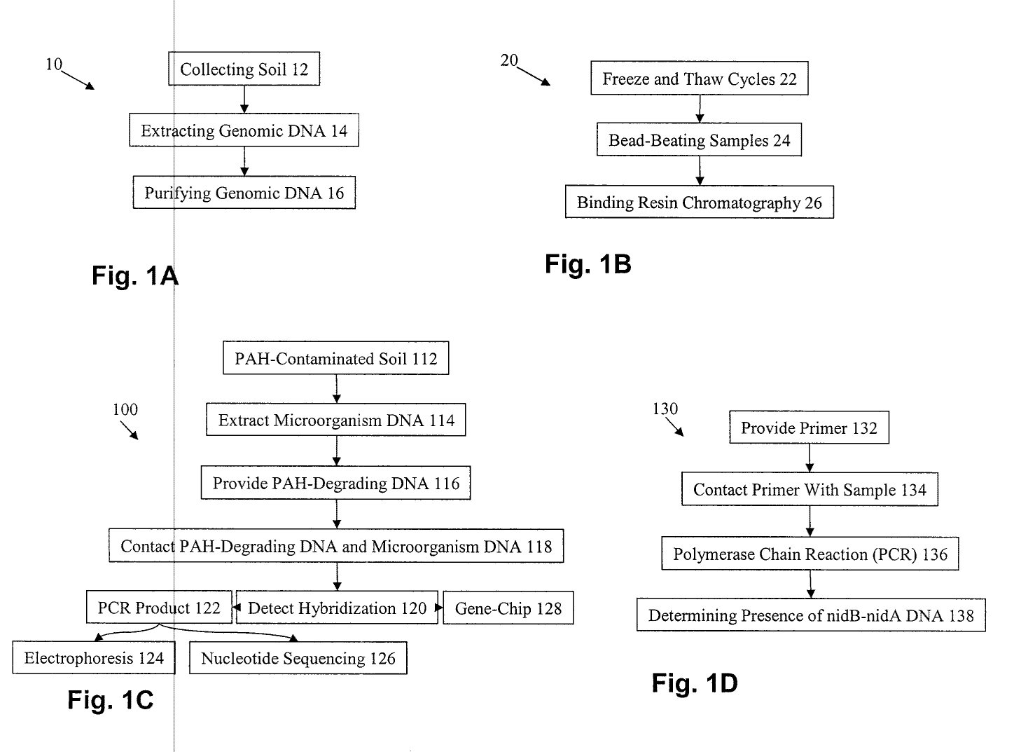

FIG. 1A is a flow diagram illustrating one embodiment of a method 10 for preparing a sample for genetic analysis. The sample preparation method 10 includes collecting soil samples (Block 12). While the soil can be collected from any location, it has been found that soil within or proximate to a site contaminated by PAHs can be a source of PAH-degrading mycobacteria that include nidB-nidA dioxygenase genes. Also, it is possible that additional strains of PAH-degrading mycobacteria can found in sites previously explored, such as the superfund site in Libby, Mont., or in sites that have not yet been explored. That is, a site that is known to be contaminated with PAHs can be a source for samples to determine whether known PAH-degrading mycobacteria are present, or a source for identifying new PAH-degrading mycobacteria.

Additionally, the sample preparation method 10 can include extracting genomic DNA from the soil (Block 14). More particularly, this can include extracting genomic DNA from microorganisms, or more preferably from mycobacteria. Extraction techniques for obtaining genomic DNA from soil are well known and described in more detail below and in the incorporated materials.

The sample preparation method 10 can also include purifying the genomic DNA (Block 16). That is, the purifying can remove impurities that can impede the ability to successfully produce a PCR product that conforms with the genome of mycobacteria present in the soil. For example, many types of proteinaceous, ionic, and hydrophobic substances can contaminate a PCR process. Purification techniques are well known and described in more detail below and in the incorporated materials.

FIG. 1B is a flow diagram illustrating one embodiment of a method 20 for preparing a sample from contaminated soil for genetic analysis. The method can include sequential freezing and thawing of the sample so that the microorganisms are also frozen and thawed in repeated cycles (Block 22). Freeze-thawing is a technique that has been shown to be effective during DNA extraction from microorganisms such as gram-positive bacteria.

Additionally, the method 20 can include bead beating the sample and microorganisms contained therein (Block 24). Bead beating usually involves mixing the sample in the presence of glass beads, and is described in more detail below and in the incorporated materials.

The method 20 can also include removing PCR inhibitors with binding resins (Block 26). This usually includes passing the sample through a chromatographic column that is comprised of various resins that can selectively either pull the genomic DNA from the sample, or pull the contaminates or PCR inhibitors from the sample so as to remove the DNA from the contaminates. Binding resin chromatography of soil samples is described in more detail below and in the incorporated references.

While various methods of sample preparation have been described herein, it is contemplated that other methods of sample preparation can be employed in accordance with the present invention. In any event, the methods of testing samples for the presence of PAH-biodegrading mycobacteria are described in more detail below.

III. Methods of Identifying PAH-Biodegrading Mycobacteria

Embodiments of the present invention include methods of identifying PAH-degrading mycobacterium that do not require culturing with PAHs, as illustrated in the flow diagram of FIG. 1C. As such, PAH-contaminated soils can be assayed for PAH-degrading microorganisms (Block 112). This is because the PAH-biodegrading mycobacterium can be identified by extracting and purifying genomic DNA directly from soil (Block 114). The genomic DNA can then be assayed for the presence of nidB-nidA dioxygenase genes indicative of the PAH-degrading mycobacterium strains JLS, KMS, and MCS as well as others. Alternatively, the purified genomic DNA can be provided from another source without performing such an extraction (i.e., when another entity has already extracted the DNA from soil and merely wants to identify the presence of PAH-degrading microbes).

In one embodiment, a method 100 for determining whether a microorganism is capable of biodegrading a PAH can be employed by assaying its genomic DNA. Such a method 100 can be performed by providing a first set of DNA molecules consisting of fragments of genomic DNA of at least one mycobacteria species capable of biodegrading a PAH (Block 116). The species can be the JLS, KMS, and MCS isolates previously identified as well as others. As such, genomic DNA of these isolates, such as the nidB-nidA dioxygenase genes, can be employed to determine whether a soil sample includes mycobacterium with substantially similar genes. The presence of these types of genes is a strong indication that the soil contains microorganisms that biodegrade PAHs.

Additionally, the method 100 can also include contacting, under hybridizing conditions, the first set of DNA molecules with a second set of DNA molecules consisting of genomic DNA of an unknown microorganism (Block 118). More particularly, it is not known whether or not the microorganism biodegrades a PAH. The contacting under hybridizing conditions can range from low, medium, and high stringency so that the ability of the probe to hybridize with the unknown genomic DNA can be modulated. This is because the stringency conditions can determine the ability of the probe (e.g., first set of DNA molecules) to properly hybridize with the genomic DNA of the unknown microorganism, which can range from partial hybridization through full hybridization where each nucleotide in the probe associates with the complement nucleotide in the genomic DNA.

The method 100 can also include detecting hybridization between the first set of DNA molecules and the second set of DNA molecules (Block 120). The detecting can include producing a PCR product (Block 122) and comparing the nucleotide sequence of the PCR product with nid dioxygenase genes by electrophoresis (Block 124), sequencing (Block 126), gene-chip, or other means. Also, a gene-chip can be used (Block 128) without performing PCR. Hybridization between the first and second sets of DNA is an indication that the microorganism is capable of biodegrading a PAH. This is because the genomic DNA of the unknown microorganism is likely to code for nidB-nidA dioxygenase enzymes when hybridization occurs. More particularly, when the first set of DNA molecules can hybridize with the second set of DNA molecules, it is likely that a nidB-nidA dioxygenase gene sequence is conserved between the known PAH-biodegrading mycobacterium and the unknown microorganism. Thus, such hybridization indicates the unknown microorganism is also a PAH-biodegrading mycobacterium.

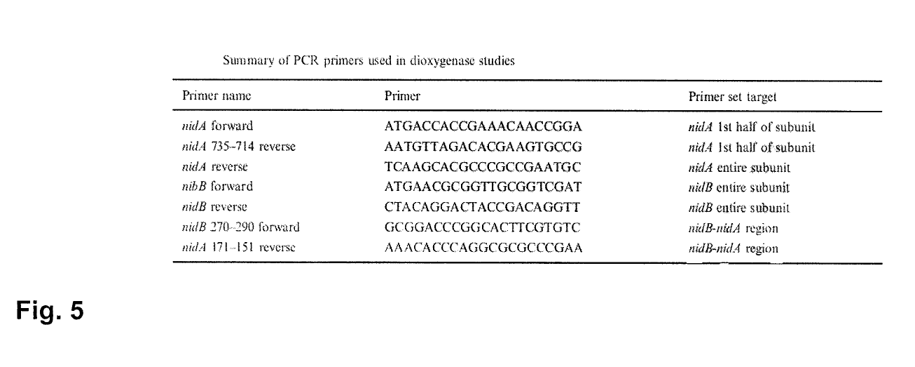

FIG. 5 is a flow diagram illustrating another embodiment of the present invention, which is a method 130 of identifying the presence of a PAH-degrading mycobacteria having nidB-nidA dioxygenase genes in a sample, such as a soil sample. Such a method 130 can be performed by providing at least one primer or primer set capable of hybridizing with a nidB-nidA dioxygenase genomic DNA nucleotide sequence of at least one known PAH-degrading mycobacteria (Block 132). As such, the preparations of primers and nucleotide sequences that can hybridize with a nidB-nidA dioxygenase genomic DNA nucleotide sequence are described in more detail below.

Additionally, the method 130 can include contacting the at least one primer or primer set with a sample (Block 134). More particularly, the contacting can be performed with a sample of genomic DNA isolated from soil as described herein, where the genomic DNA has been purified so that it can be used in PCR. This is because substances in an unpurified sample, such as proteinaceous or other substances, can contaminate the PCR and result in inaccurate PCR products. In any event, the method 130 can be favorable when it is not known whether or not the sample includes a PAH-degrading mycobacterium. In part, this is because a sample known to have PAH-degrading microorganism may not need to be assayed as described.

The method 130 can also include producing a PCR product (Block 136). A PCR product can be produced by any of the well-known PCR methods as well as those subsequently developed. Briefly, PCR products can be obtained by annealing the primer to genomic DNA complement thereto and introducing a polymerase capable of adding nucleotides to the primer so as to become a complement of the genomic DNA to which it has annealed. More details on producing PCR products are provided below and in the incorporated materials.

Also, the method 130 can include determining whether the PCR product indicates the presence of genomic DNA of a microorganism having a nidB-nidA dioxygenase nucleotide sequence in the sample (Block 138). Such a determination can be made when the PCR product is similar to known nidB and/or nidA dioxygenase gene sequences. That is, when the PCR product is comparable to known nidB and/or nidA nucleotide sequences, it is indicative that the sample, such as a soil sample or sample prepared therefrom, includes a mycobacterium capable of degrading PAHs. In any event, additional details on such a determination are provided herein.

In accordance with the foregoing, the present invention can also include a method of identifying the presence of PAH-degrading mycobacterium by using selected sequences of genomic DNA identified in the Sequence Listing. Such a method can be used during a process of determining whether or not a soil sample contains PAH-degrading mycobacteria as described herein or elsewhere. The method can include producing a nucleotide sequence which consists of at least a sequential portion of at least one of SEQ ID NO: 1 (Block 144), SEQ ID NO: 3 (Block 146), SEQ ID NO: 5 (Block 148), SEQ ID NO: 7 (Block 150), SEQ ID NO: 9 (Block 152), SEQ ID NO: 11 (Block 154), or any complement thereof. Alternatively, an embodiment of the present invention is a nucleotide sequence consisting of the foregoing sequence identification numbers. The nucleotide sequences of such sequence identification numbers are described below in the Sequence Listing.

In one embodiment, any of the foregoing methods can include performing a PCR to amplify the amount of a second set of DNA molecules (e.g., DNA isolated from soil) as at least a portion of the method for determining whether a microorganism is capable of biodegrading PAHs or whether a sample includes such microorganisms. As such, a first portion of a first set of DNA molecules (e.g., PAH-degrading mycobacteria genomic DNA molecules) includes a plurality of primers. That is, primers, primer sets, and/or primer pair can be prepared from PAH-degrading mycobacteria genomic DNA molecules. Each of the primers can be comprised of a primer nucleotide sequence having from about 8 to about 30 nucleic acids, more preferably from about 19 to about 25, and most preferably about 21 nucleic acids. In any event, the primers hybridize with at least a sequential portion of at least one of SEQ ID NOs: 1, 3, 5, 7, 9, 11, or any complements thereof. Alternatively, the primers hybridize with a conserved sequential sequence found in at least three of SEQ ID NOs: 1, 3, 5, 7, 9, 11, or any complements thereof. Also, the method can include the preparation of such primers.

Sequence identification numbers 1, 3, and 5 are coding sequences of the nidA genes from the JLS, KMS, and MCS strains, respectively. Additionally, sequence identification numbers 7, 9, and 11, are coding sequences of the nidB genes from the JLS, KMS, and MCS strains, respectively.

In one embodiment, any of the foregoing methods can include primers that hybridize with conserved sequences of the JLS, KMS, and MCS genomic DNA. More particularly, at least one primer sequence of a plurality of primers hybridizes with a conserved nucleotide sequence in each of SEQ ID NOs: 1, 3, and 5, or complements thereof. Additionally, at least one of the primer sequences hybridizes with a conserved nucleotide sequence in each of SEQ ID NOs: 7, 9, and 11, or complement thereof.

Consistent with the foregoing, one embodiment of a method includes primers selected from the group consisting of SEQ ID NOs: 26, 27, 28, 29, 30, 31, 32, 33, 34, and 35 or any complements thereof. The sequence identification numbers 26-35 are primers for use in PCR amplification of mycobacteria genomic DNA. Alternatively, one embodiment of the present invention specifically excludes the use of primers selected from the group consisting of SEQ ID NOs: 26, 27, 28, 29, 30, 31, 32, 33, or any complement thereof. Additionally, other primer sequences prepared from conserved regions within the genomic DNA of the JLS, KMS, and/or MCS isolates can be prepared and used in accordance with the present invention.

Consistent with the foregoing, one embodiment of a method includes primers selected from the group consisting of SEQ ID NOs: 36, 37, 38, 39, 40, and 41, or any complements thereof. The sequence identification numbers 26-35 are primers for use in PCR amplification of mycobacteria genomic DNA. Alternatively, one embodiment of the present invention specifically excludes the use of primers selected from the group consisting of SEQ ID NOs: 36, 37, 38, 39, 40, and 41, or any complement thereof. Additionally, other primer sequences prepared from conserved regions within the genomic DNA of the JLS, KMS, and/or MCS isolates can be prepared and used in accordance with the present invention.

Optionally, an embodiment of the present invention can include a method that contacts a second portion of the first set of DNA molecules (e.g., nidB promoter sequence) with the molecules consisting of genomic DNA of the unknown microorganism (e.g., second set of DNA molecules). In this optional embodiment, the second portion of the first set of DNA molecules includes a second plurality of primers, wherein each of the primers in the second plurality is comprised of a primer nucleotide sequence having from about 8 to about 30 nucleic acids, more preferably about 19-25, and most preferably about 21 nucleic acids. The primers can also hybridize with a portion of SEQ ID NOs: 13, 14, 15, 16, and 17, or complement thereof. Additionally, the primers can hybridize with a conserved nucleotide sequence within SEQ ID NOs: 13, 14, 15, 16, and 17, or complement thereof. These sequence identification numbers correspond with nidB promoter sequences of mycobacteria strains JLS, KMS, MCS, Flav, and Pyr-1 strains, respectively.

In another embodiment, the present invention can include a method that contacts a second portion of the first set of DNA molecules (e.g., nidB promoter sequence) with the molecules consisting of genomic DNA of the unknown microorganism (e.g., second set of DNA molecules). In this optional embodiment, the second portion of the first set of DNA molecules includes a second plurality of primers, wherein each of the primers in the second plurality is comprised of a primer nucleotide sequence having from about 8 to about 30 nucleic acids, more preferably about 19-25, and most preferably about 21 nucleic acids. The primers can also hybridize with a portion of SEQ ID NOs: 42 and 43, or complement thereof. Additionally, the primers can hybridize with a conserved nucleotide sequence within SEQ ID NOs: 42 and 43, or complement thereof. Further, the primers can consist of nucleotide sequences within SEQ ID NOs: 42 and 43, or complement thereof. These sequence identification numbers correspond with nidB promoter sequences of mycobacteria strains JLS, KMS, and MCS.

In another optional embodiment, the present invention can include a method that contacts a third portion of the first set of DNA molecules (e.g., 16S ribosomal gene promoter sequence) with the molecules consisting of genomic DNA of the unknown microorganism. In this optional embodiment, the third portion of the first set of DNA molecules includes a third plurality of primers, wherein each of the primers in the third plurality is comprised of a primer nucleotide sequence having from about 8 to about 30 nucleic acids, more preferably about 19-25, and most preferably about 21 nucleic acids. The primers can hybridize with a portion of SEQ ID NOs: 18, 19, 20, 21, and 22, or complement thereof. Also, the primers can also hybridize with a conserved nucleotide sequence within SEQ ID NOs: 18, 19, 20, 21, and 22, or complements thereof. These sequence identification numbers correspond with a promoter sequence for 16S ribosomal genes of mycobacteria strains JLS, KMS, MCS, Flav, and Pyr-1 strains, respectively.

Still another optional embodiment includes a method that uses a mycobacterium 16S ribosomal gene to identify whether or not the sample includes, or organism is, a mycobacterium. Such a method includes the following: providing at least one primer capable of hybridizing with a mycobacterium 16S ribosomal DNA nucleotide sequence; hybridizing the at least one primer with the 16S ribosomal DNA nucleotide sequence; producing a polymerase chain reaction product; and determining whether the polymerase chain reaction product indicates the presence of PAH-degrading mycobacteria. Also, the 16S ribosomal DNA nucleotide sequence can be a portion of the promoter sequence.

Moreover, the primers for use in the present invention can include or consist of at least a portion of the genomic DNA of mycobacteria strains JLS, KMS, MCS. That is, the primers can have nucleotide sequences that are included within the genomic DNA of mycobacteria strains JLS, KMS, MCS. Alternatively, the primers can have the nucleotide sequence, or portion of nucleotide sequence, of any of the nucleotide sequences in the Sequence Listing. As such, any sequential nucleotide sequence of the sequences in the Sequence Listing can be used as a primer as described herein.

Additionally, methods of identifying the presence of PAH-degrading mycobacteria having nidB-nidA dioxygenase genes in a sample, or determining whether a microorganism is capable of biodegrading a PAH in accordance with the present invention can include various techniques. These techniques can include various assays or experimental protocols that compare the genomic DNA found in a sample or unknown microorganism with the genomic DNA of known PAH-biodegrading mycobacteria. In part, this is because the inventors have discovered that PAH-biodegrading organisms are often mycobacteria having conserved genetic codes for nidB-nidA dioxygenase genes.

In one embodiment, the techniques for the identification or determination of contaminant-degrading mycobacteria can include performing PCR on the purified soil samples. This includes the use of primers that hybridize with genomic DNA of known contaminant-degrading mycobacteria and/or the nidB-nidA dioxygenase genes included therein, or primers that have a sequential nucleotide sequence found within the genomic DNA of known contaminant-degrading bacteria and/or nidB-nidA dioxygenase genes. In any event, a soil sample or microorganism can be determined to be PAH-degrading mycobacteria from the PCR product. Techniques for performing PCR are well known and described in more detail below and in the incorporated materials.

In one embodiment, the determination of whether or not known genomic DNA indicates the presence of contaminant-degrading mycobacteria can include comparing the size of the PCR product with a DNA ladder by performing electrophoresis. Also, electrophoresis can compare the PCR product with known nidB DNA, nidA DNA, and/or combinations thereof. Furthermore, electrophoresis can use known nidB DNA and/or nidA DNA from at least one of JLS, KMS, or MCS.

In one embodiment, the determining of whether or not known genomic DNA indicates the presence of a contaminant-degrading mycobacterium can include sequencing the PCR product to determine the nucleotide sequence thereof. Sequencing is an established and well-known technique that provides the sequence of the nucleic acids. Additional information on sequencing protocols can be found in the incorporated materials and elsewhere.

In any event, after the sequence of the PCR product is obtained, the sequence can be compared with a known contaminant-degrading mycobacterium nidA and/or nidB nucleotide sequence. Also, the sequence can be compared with a nidA and/or nidB nucleotide sequence from a known PAH-degrading mycobacterium selected from the group consisting of JLS, KMS, MCS, Mycobacterium vanbaalenii, Mycobacterium frederiksbergense strain FAn9T, Mycobacterium flavescens strain PYR-GCK. Also, it is contemplated that the PCR product sequence can be compared to future-discovered contaminant-degrading mycobacterium genomic DNA sequences.

In one embodiment, comparing the PCR product nucleotide sequence with a known PAH-degrading mycobacterium nidA and/or nidB nucleotide sequence can result in a substantially homologous or conserved nucleotide sequence between the unknown mycobacteria and the known PAH-degrading mycobacterium. As such, a nucleotide identity match greater than 95% indicates the sample, such as a soil sample or microorganism sample, contains PAH-degrading mycobacteria. More particularly, the nucleotide identity match is greater than 97% and most preferably 99% or greater.

### EXAMPLES OF EMBODIMENTS OF THE INVENTION

The following examples illustrate embodiments of the present invention that can be employed in order to determine whether or not a soil sample includes PAH-biodegrading mycobacteria. As such, Examples 1-2 were performed to identify whether or not PAH-contaminated soils contained PAH-degrading microorganisms. After PAH-microorganisms were found, Example 3 was performed to determine the taxonomic classification of the microorganisms so that experiments could be designed to identify the mechanism for PAH-degradation. Examples 4-5 were performed to compare the use and degradation of PAHs with known the known PAH-degrader M. vanbaalenii. Subsequently, after these experiments were conducted and the general taxonomic classification of the PAH-degraders were determined by Example 6 to be mycobacteria, PCR analysis of the genetic material was performed by preparing primers to assess whether the nid dioxygenase enzyme genes were conserved in the mycobacterial isolates.

The discovery that the PAH-degrading mycobacterial isolates included some conserved regions in the nid dioxygenase genes lead to the present invention that includes methods of assessing PAH-biodegradation capabilities directly from a soil Z >Ad sample. As such, Examples 7-9 were performed on soil extracts without having to undergo experiments similar to those described in Examples 1-6. However, while Examples 1-6 were previously used to isolate and identify PAH-degrading mycobacteria, such Examples 1-6 can now be useful with present invention as confirming studies rather than identification assays. That is, after a soil sample is determined to include PAH-degrading mycobacteria by PCR of DNA extracted directly from soil, Examples 1-6 can be performed to confirm various characteristics of the PAH-degrading bacteria.

### Example 1

An example of soil identified to include PAH-degraders includes the PAH-contaminated soil from the land treatment unit (LTU) at the Champion International Superfund Site in Libby, Mont. The soil was characterized as a loam (48% sand, 39% silt and 13% clay). The soil had a pH of 6.6, an EC of 4.5 mhos/cm, and 1.88% organic carbon. The soil was passed through a 1.7 mm sieve and homogenized by hand and was stored in the dark at 4° C. until it was used. The soil had a moisture content of 10.2%. As such, it contemplated that various other types of soil can also include PAH-degraders.

### Example 2

The LTU soil was processed in order to assess the presence of PAH-degraders. Briefly, colonies capable of degrading pyrene were obtained from the LTU soil by suspending samples (0.1 g/ml) in sterile distilled water followed by serial dilution and spreading onto a basal salts medium (BSM) containing mineral nutrients but no carbon source. The basal salts medium contained (in 1 liter): 2.38 g (NH₄)SO₄, 0.28 mg FeSO₄*7H₂O, 10.69 mg CaCl₂*7H₂O, 0.25 g MgSO₄*7H₂O, 0.50 g NaCl, 1.42 g Na₂HPO₄, 1.36 g KH₂PO₄, pH 6.5. Agar was added at 1.5%. The plates were airbrushed with a solution of pyrene in hexane/acetone (1:1) until an opaque layer had formed on the surface. The inoculated plates were placed in an incubator at 30° C. and bacteria were allowed to form visible colonies. The colonies producing a clear zone in the opaque layer were transferred to tryptic soy agar plates for single colony isolation. Four types of bacteria isolates were initially isolated using this technique, three of which were used for subsequent studies. For storage, cells of these three bacteria, the JLS, KMS, and MCS strains, were grown in Luria broth (LB) (Difco, Becton Dickinson, Sparks, Md.) cultures and were suspended in 15% glycerol before being stored at −80° C. Liquid media cultures were generated from freezer stocks in BSM+ (9:1 mixture v/v of BSM and LB) by shaking at 220 rpm at 25° C. Five-day-old cultures were used for analysis and various inoculations. Additionally, utilization of phenathrene and benzo(a) pyrene by isolates JLS, KMS, and MCS was determined using BSM plates possessing an overlay of these materials as described above.

The results indicated that the three bacteria from the PAH-contaminated LTU soil formed PAH-degrading bacterial colonies surrounded by zones of clearing of pyrene layered onto BSM-agar plates. Each isolate grew rapidly in broth culture on LB media. All three isolates were gram positive, although they had different cell morphologies. Isolate JLS was a coccus, KMS a short rod and MCS a long rod.

### Example 3

The JLS, KMS, and MCS strains were initially identified by fatty acid analysis of the single colony isolates as performed by MIDI (PA) and by partial analysis of the 16S ribosomal gene sequences. Fatty acid composition of the isolates (MIDI) identified them as mycobacterium isolates but without a close match to other species in the existing MIDI database.

Based on this initial identification, PCR primers were synthesized with homology to the 16S ribosomal DNA within the genus Mycobacterium. The 16S ribosomal gene sequences were analyzed by preparing primers and producing a PCR product with such primers. Briefly, 10 ml of cells from liquid cultures were harvested by centrifugation and washed twice with sterile phosphate-buffered saline (PBS), pH 7.4 and once with sterile distilled water. Cells were re-suspended in 1 ml of 10 mM Tris-HCl (pH 8.0), 1 mM EDTA, and 1% Triton X-100 (TET). The suspension was subjected to five cycles of freezing for 3 min in liquid nitrogen and 1 min heating in boiling water. After the last boiling step, the suspension was placed on ice to obtain the lysis material used directly in subsequent PCR processes.

Primers for PCR analysis of the complete 16S ribosomal gene were generated based on conserved sequences. Three forward primers (Myc16-1, Myc16-3, and Myc16-5) and three reverse primers (Myc16-2, Myc16-4, and Myc16-6) were generated to permit the synthesis of three PCR products of approximately 500 bp in size to cover the entire 16S ribosomal gene. These primers were: Myc16-1 (SEQ ID NO: 36), 5′-TGGAGAGTTTGATCCTGGCTC-3′; Myc16-2 (SEQ ID NO: 37), 5′-CGGCTGCTGGCACGTAGTTG-3′; Myc16-3 (SEQ ID NO: 38), 5′-TCGGGTTGTAAACCTCTTTC-3′; Myc16-4 (SEQ ID NO: 39), 5′-GACGA CAGCCATGCACCACC-3′; Myc16-5 (SEQ ID NO: 40), 5′-GGGTTTGACATGCACAGGAC-3′; Myc16-6 (SEQ ID NO: 41), 5′-TACCTTGTTACGACTTCGTCC-3′. PCR reactions were conducted in an Ericomp TwinBlock thermocycler. The reaction mixtures contained 5 μl of mycobacterial cell lysate, 6 mM MgCl₂, 200 nM PCR primers, and 1 mM dNTPs in 1 x reaction buffer (MBI Fermentas, Inc., Amherst, N.Y.). The reaction mixture was held at 94° C. for 7 min prior to the addition of 0.5 units of Taq polymerase. PCR reactions were cycled 35 times through 94° C. for 1 min, 55° C. for 2 min, and 72° C. for 2 min. QIAquick gel purification kits (QIAGEN Inc., Valencia, Calif.) were used to purify the PCR products, which were subsequently sequenced.

Nucleotide sequencing was performed by automated sequencing using dye-labeled universal terminators on an Applied Biosystem (ABI) 373 DNA sequencer (Foster City, Calif.). Nucleotide sequences were obtained from both strands of each PCR product and each sequence was confirmed with at least two separate sequencing reactions for all products.

The 16S ribosomal gene nucleotide sequences from mycobacterium species were aligned using BLAST (National Center for Biotechnology Information). 16S ribosomal nucleotide sequences were transferred into PAUP format and analyzed using the PAUP version 4.0b4 (Swofford 1998) analysis program. Alignment gaps were treated as absent and all sites were weighted equally. Branch lengths are proportional to the number of inferred nucleotide substitutions. The branch-and-bound algorithm, with consensus tree options retaining groups with frequency >50%, was used to infer maximum parsimony trees. Clade stability was assessed by 1000 bootstrap replications. Tree lengths, branch lengths, consistency indices (CI) and retention indices (RI) were calculated with PAUP version 4.0b4 (Swofford 1998). The sequences for the 16S ribosomal gene for the Mycobacterium sp. have been entered into GenBank: AF387804 for JLS, AY083217 for KMS, and AF387803 for MCS.

Nucleotide sequence analysis of the PCR products from each strain showed that the isolates KMS and MCS were identical over the entire 1394 bases of their 16S ribosomal gene, whereas the sequence from JLS differed from KMS and MCS by two nucleotides (see, incorporated references).

### Example 4

The catalase and SOD isozyme patterns of the JLS, KMS, and MCS isolates were assayed for further identification and classification. To prepare extracts for enzyme activity analysis, cells were grown in a 9:1 (V/V) mixture of BSM and LB for three days shaking at 220 rpm and 28° C. Cells were harvested by centrifugation at 3,000 g, re-suspended in 0.3 ml of 50 mM potassium phosphate buffer, pH 7.8, and sonicated three times each for 15 s. Sonicates were centrifuged at 13,000 g for 15 min to obtain the supernatants used in the assays. Non-denaturing 7.5% polyacrylamide gels were run and stained to detect catalase and SOD isozymes as described in the incorporated materials. Protein concentrations in cell extracts were estimated using the bicinchoninic acid (BCA) protein assay kit (Pierce Co., Rockford, Ill.).

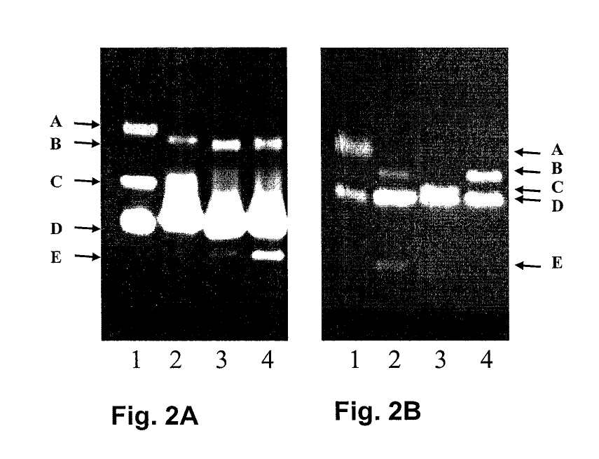

The catalase and SOD isozyme profiles for stationary-phase cells of JLS, KMS and MCS grown in BSM+ are shown in FIGS. 2A-2B along with the profiles from M. vanbaalenii for comparison. FIGS. 2A-2B illustrates a comparison of catalase (FIG. 2A) and superoxide dismutase (FIG. 2B) isozyme profiles of PAH-degrading mycobacterium isolates JLS (Lane 1), KMS (Lane 2), and MCS (Lane 3) with Mycobacterium vanbaalenii (Lane 4). Various isozymes are indicated as A-E.

The isozymes have been assigned different letters according to their relative motilities. Multiple catalase activities were detected with each isolate. One band was dominant with isolates KMS and MCS and vanbaalenii. The mobility of the major band in KMS was distinct from that of MCS and its pattern most resembled that of M. vanbaalenii. The mobility of minor bands with catalase activity further distinguished isolates KMS, MCS, and vanbaalenii. Isolate JLS had a very distinct band pattern where no one activity was dominant. Similarly, the SOD isozyme profiles were distinct between all four isolates. One SOD band was common to all four isolates. The SOD isozyme pattern from vanbaalenii was more similar to KMS than MCS.

### Example 5

PAH-utilization and pyrene-mineralization experiments were conducted to determine the ability of the JLS, KMS and MCS isolates to biodegrade PAHs. Briefly, microcosms were established using one gram of native or sterilized PAH-contaminated soil from the LTU-2, in a 7 ml vial. The soil had been amended 24 hours previously with 200 μof 500 ppm pyrene (dissolved in methanol) with or without a supplement of 2.14 μCi of [4, 5, 9, 10-¹⁴C]-pyrene (specific activity of 56 mCi/mmol). The total DPM in each vial was 117167. Inoculate of 10⁸ colony forming units (CFU) in 200 μl of the mycobacterium isolates JLS, KMS, MCS and vanbaalenii, were added to test vials. The cells had been grown for five days to stationary-phase in BSM+, pelleted, and washed with sterile distilled water. Other soils were treated with 200 μl of water as a control. For certain controls, viable microbes in the soil were killed by adding 1 mg mercuric chloride dissolved in 0.2 ml methylene chloride added to 1 g soil and incubating overnight at room temperature before use. Each vial was placed singly into 100 ml reaction vessels containing another vial with 1.0 ml 0.1N KOH to trap any released carbon dioxide. The trapping solution was removed at defined times and assayed for ¹⁴C by scintillation counting. Also at defined times, CFU were assessed from the microcosms containing only unlabeled pyrene. Approximately 0.1 g of soil was removed and added to 1 ml of sterile water. The mixture was vortexed for 10 s and then serially diluted and plated on LB agar plates. Colonies were counted after 2 days of incubation at 25° C.

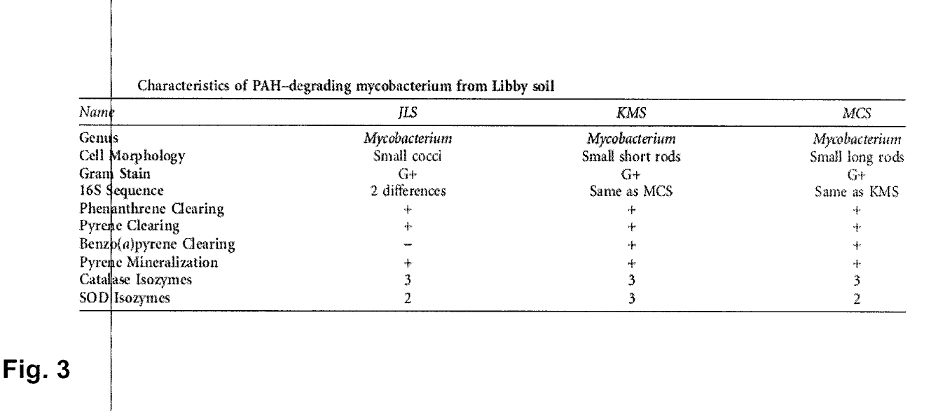

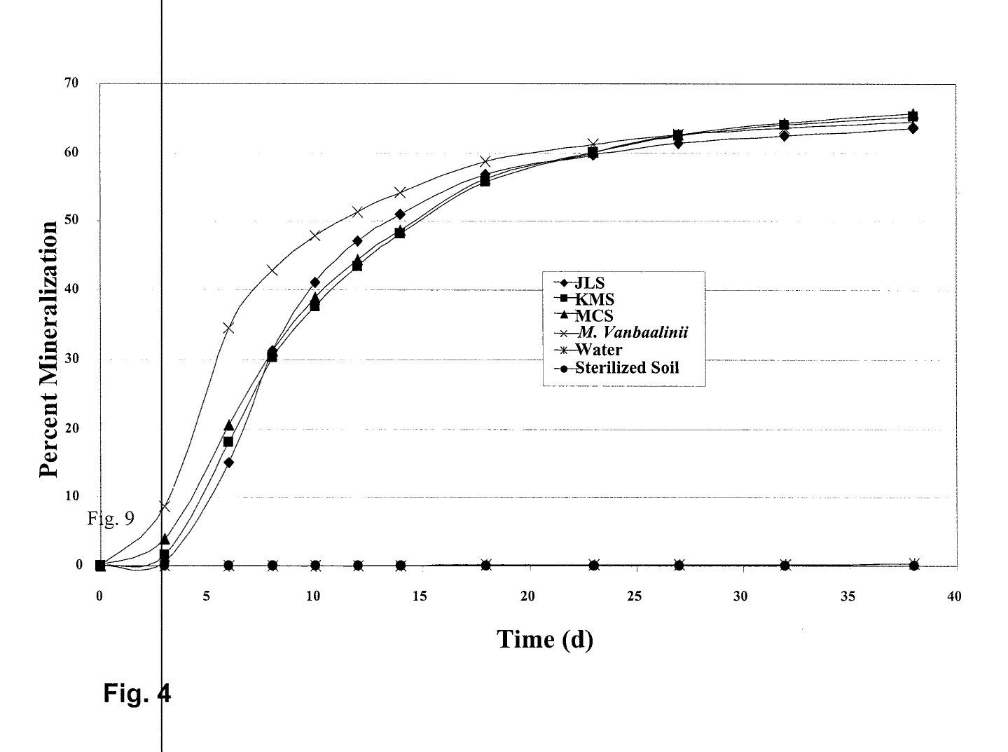

The JLS, KMS, and MCS isolates cleared BSM plates overlayed with phenanthrene, whereas only KMS and MCS, but not JLS, degraded benzo(a)pyrene as shown in FIG. 3. Equal mineralization of ¹⁴C-pyrene was demonstrated by the three strains after inoculation of the native Libby LTU soil, as shown in FIG. 4. Mineralization of pyrene was observed by day three and although the initial rate of mineralization was faster with M. vanbaalenii than with JLS, KMS, and MCS (significantly different up to 7 d), the final extent of mineralization was similar among all isolates. After 18 days, over 55% of the labeled pyrene was mineralized and by 38 days approximately 65% of the pyrene was mineralized. Neither the non-inoculated LTU soil nor the soil treated with HgCl₂ showed mineralization in this time frame. In an extended time study, significant (˜7%) mineralization by the indigenous microbes in the dried LTU soil required at least 90 days (data not shown). Colony forming units were monitored throughout the mineralization experiments. After 38 days, CFUs for each of the Mycobacterial isolates were at 10⁷ CFU/g soil. While the HgCl₂ poisoned soil had no detectable CFUs, the non-inoculated water controls showed background levels of CFUs at around 10⁶ CFU/g soil at 38 days.

### Example 6

After the JLS, KMS, and MCS isolates were determined to be mycobacteria with PAH-degrading capabilities, experiments were conducted to identify whether or not certain nidB-nidA dioxygenase genes were conserved in relation to the known Mycobacterium vanbaalenii. Primers for PCR analysis of dioxygenase genes, nidA and nidB, were generated based on the Mycobacterium vanbaalenii sp. nov. nidA and nidB sequences. The nidA specific primers were NidAF (SEQ ID NO: 26): 5′-ATGACCACCGAAACAACCGGA-3′, NidAR(SEQ ID NO: 27): 5′-TCAAGCACGCCCGCCGAATGC-3′, NidAF642-663 (SEQ ID NO: 28): 5′-GCCGCCGACAATTTTGTCGGC-3′ and NidAR735-714 (SEQ ID NO: 29): 5″-GAAGTTTGGGTCACCGGGCGC-3′. The nidB specific primers were NidBF (SEQ ID NO: 30): 5′-ATGAACGCGGTTGCGGTCGAT-3′, NidBR (SEQ ID NO: 31): 5′-CTACAGGACTACCGACAGGTT-3′, NidBF207-228 (SEQ ID NO: 32): 5′-GGTTTGGAGATGCGGGTCCTT-3′, and NidBR297-276 (SEQ ID NO: 33): 5′-AATGTTAGACACGAAGTGCCG-3′. PCR conditions for the dioxygenase gene analysis were the same as describe above in Example 3, except PCR reactions were cycled 35 times through 94° C. for 1 min, 55° C. for 1 min, and 72° C. for 1 min and were performed in an Eppendorf Mastercycler gradient PCR machine (Eppendorf, N.Y.). Clustal W multiple sequence alignment method parameters were: weight matrix of IUB/BESTFIT, gap penalty of 15.00 and gap extension penalty of 6.66. Multiple alignment parameters were: DNA transition weight of 0.5, weight matrix of IUB/BESTFIT, gap penalty of 15.00 and gap extension penalty of 6.66, and decay divergent sequences of 30 (Clustal W ret).

The JLS, KMS, and MCS were determined to have nidB-nidA genes based on the analysis of the PCR products. Using the NidAF/NidAR and NidBF/NidBR primer sets, PCR products of approximately 1368 bps and 510 bps were produced, respectively. Sequence analysis of these PCR products demonstrates that both of these genes are highly homologous to previously isolated dioxygenase genes from PAH-degrading mycobacterium. PCR reactions using NidAF642-663 and NidBR297-276 yielded no PCR products, while PCR reactions with NidBF207-228 and NidAR735-714 did generate PCR products.

These results indicate that the nidB and nidA genes are adjacent to each other in the sequence nidB followed by nidA (e.g., nidB-nidA sequence), an order that is unique to the PAH-degrading mycobacterium. Phylogenetic analysis of the sequences for nidB shows that the sequences from the Libby isolates cluster together but are most similar to sequences from other PAH-degrading mycobacteria. There is greater than 97% identity of the nucleic acid sequences of the nidB genes among the JLS, KMS, and MCS isolates, M. vanbaalenii, M. frederiksbergense, M. flavescens, and M. gilvum, all which have the unique nidB-nidA sequence of dioxygenase genes. On the other hand, C. testosteroni, Nocardiodes. KP7, and Rhodocoiccus. NCIMB12038 have less than 60% identity of the nidB genes and have the more common dioxygenase gene order of nidA followed by nidB.

Phylogenetic analysis of nidA demonstrates that like nidB, the nidA sequences for the JLS, KMS, and MCS isolates cluster with other PAH-degrading mycobacteria. While the nidA gene for JLS and KMS are greater than 99% identical to each other and the other mycobacteria in the analysis, the MCS nidA sequence is more divergent. The MCS nidA gene is 96% identical to each of the other mycobacteria, including the KMS and JLS isolates. Divergence in the MCS sequence is primarily located in two locations: a nine-nucleotide insertion at nucleotide position 21 and a series of differences between nucleotide positions 300 and 350.

### Example 7

A rapid assay for determining whether or not PAH-contaminated soil includes PAH-degrading mycobacteria includes a protocol for extracting mycobacterial DNA directly from soil without culturing. Briefly, DNA was extracted from PAH-contaminated soil using combined freeze-thaw cycling, vortexing with glass beads, and chromatographic removal of PCR inhibitors in PCR inhibitor binding resins. The materials used for this procedure were obtained from the Ultra Clean™ Soil DNA Isolation Kit from MoBio® Laboratories, Inc. (Solana Beach, CALIF.), and the Soil Master™ DNA Extraction kit from Epicentre® (Madison, Wis.). The procedures used were a combination of standard protocols for these kits with some modifications.

Soil samples (0.30 g wet weight) were placed in the Ultra Clean™ Soil DNA Isolation Kit bead beater tubes. The tubes were placed at −80° C. for 10 min and then immediately thawed in a water bath at 60° C. The tubes were secured to a vortex machine and vortex mixed for 10 min at maximum speed. They were then centrifuged at maximum speed for 30 s, the supernatant was placed in a clean microcentrifuge tube, centrifuged again for 30 s, and the supernatant was placed in a clean microcentrifuge tube. One ml of cold (−20° C.) isopropyl alcohol was added to precipitate the nucleic acids. Following incubation at 6° C. for 10 min, the tubes were centrifuged for 10 min at maximum speed. The alcohol was decanted and the pellet was suspended in 180 μL of sterile deionized water. Residual protein was precipitated and inhibitors removed using the standard protocol for the Soil Master™ DNA Extraction kit from Epicentre® starting with the protein precipitation step (Epicentre 2003). The final purified DNA pellet was suspended in 50 μL of TE buffer.

### Example 8

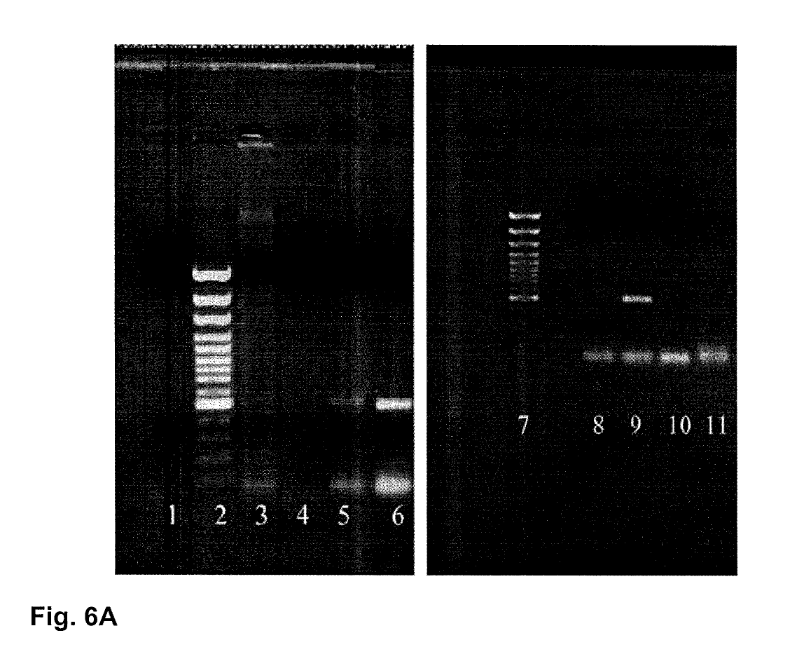

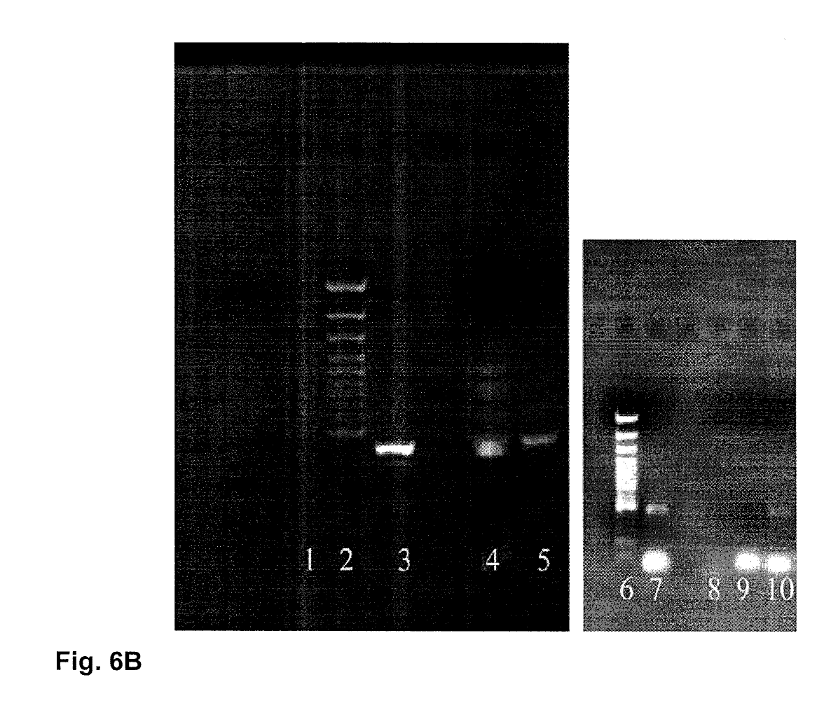

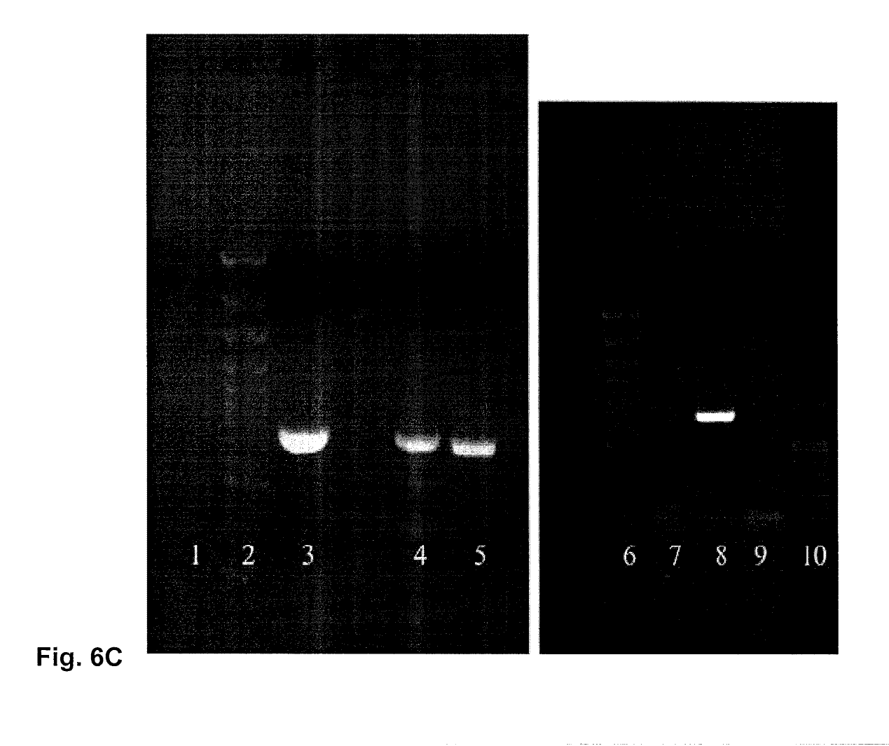

After the DNA was extracted from the PAH-contaminated soils, PCR products were prepared to determine if the DNA included the nid dioxygenase genes, which is a strong indication of the presence of PAH-biodegrading mycobacteria. Briefly, PCR oligonucleotide primers were ordered from Qiagen Operon (Alameda, Calif.), and all PCR reagents were purchased from MBI Fermentas, Inc. (Amherst, N.Y.). All PCR was performed using an Eppendorf Mastercycler® thermocycler and 0.20-ml tubes. PCR primers were designed based on homology between the known Mycobacteria nidB-nidA dioxygenase genes conserved in the JLS, KMS, and MCS mycobacteria. The primers were designed to target four regions on the mycobacteria genome: the entire nidA gene, the first half of the nidA gene, the entire nidB gene and a section of the genome constituting part of the nidB and nidA genes and including the region between the genes, as illustrated in FIG. 6.

The PCR was performed as in Example 6, with some exceptions. Briefly, the PCR cycling conditions were as follows: a four minute hot start at 94° C.; cooling to 4° C. for addition of Taq polymerase; 40 cycles of 94° C. denaturation for one minute; 60° C. annealing; 72° C. lengthening for one minute (2 minutes for the nidA forward and reverse primers); at the end of cycling 72° C. for 5 min and storage at 4° C. The reagent concentrations were 27.5-29.5 μL sterile deionized water, 5 μL of 10X PCR buffer with and without ammonium sulfate, 2 μL of 1 mM dNTP, 8 μL of 25 mM MgCl₂ solution, 2 μL of each primer solution, 0.5 μL of Taq polymerase (diluted in 9.5 μL of water prior to addition), and 3 μL of template solution. Templates were isolated genomic DNA, cell cultures grown under conditions stated above, or DNA soil extractions. All PCR experiments included isolated purified genomic DNA from JLS, MCS, or KMS as positive controls and blank samples without template added as negative controls on the PCR reaction. All PCR products were observed using electrophoreses of the PCR products on 1.1% agarose gels stained with ethidium bromide. The gels were marked by loading 7 μL of Gene Ruler™ 100 bp DNA Ladder Plus (0.1 mgDNA/ml) from MBI Fermentas.

When mycobacteria genomic DNA was used as a template, the primer set targeting the first half of nidA produced product 735 bp long in JLS, KMS, and M. vanbaalenii and 744 bp in MCS. The primer set targeting the entire nidA gene produced products 1368 bp in JLS, KMS, and M. vanbaalenii, and 1377 bp in MCS. The nidB primer set produced a product 510 bp long, and the nidB to nidA primer set produced a product 471 bp long in JLS, KMS, and M. vanbaalenii, and 480 bp in MCS. The nidA and nidB primer sets were designed from homology with M. vanbaalenii and the length of the products was confirmed with the sequencing of JLS, KMS, and MCS.

### Example 9

The PCR products obtained from Example 8 were sequenced and compared to known nidB-nidA dioxygenase gene sequences. Briefly, nucleotide sequencing was performed by automated sequencing using dye-labeled universal terminators on an Applied Biosystem (ABI) 373 DNA sequencer (Foster City, Calif.). Nucleotide sequences were obtained from both strands of each PCR product. The dioxygenase sequences were aligned using BLAST (National Center for Biotechnology Information). These aligned sequences were then compared to the nidB and nidA genes. These nid genes included nidA genes from M. gilvum strain BB1, M. frederiksbergense strain FAn9T, M. flavescens strain PYR-GCK, M. vanbaalenii, JLS, MCS, KMS. These nid genes included nidB genes from M. gilvum strain BB1, M. frederiksbergense strain FAn9T, M. flavescens strain PYR-GCK, M. vanbaalenii, JLS, MCS, KMS. Substantial regions of the nid genes (over 400 bp in the nidB and over 700 bp in the nidA) were compared and the percentage presented in the results is the number of nucleotide matches.

The nid dioxygenase primers tested on LTU soils indicated that DNA extracted directly from soil can be purified and amplified by PCR in order to determine whether or not the soil contains PAH-biodegrading mycobacteria. The lack of contamination in the extraction and in the PCR reagents was verified by including PCR reactions without template added, and PCR reactions with extractions from sterilized clean sand as template. Neither of these samples produced products with any of the primer sets. The results of the PCR on soil extracts indicate that the nidB-nidA dioxygenase gene probe detected nidB-nidA dioxygenase genes in the LTU2 soil by electrophoresis, as shown in FIGS. 6A-6C.

FIG. 6A is a gel electrophoresis of the soil gene probe targeting nidB region. Lane 1 is sterile sand, lane 2 is 100 bp DNA ladder plus, lane 3 is JLS, lane 4 is PCR blank, lane 5 is LTU soil extraction, lane 6 is LTU soil extraction, lane 7 is 100 bp DNA ladder plus, lane 8 is PCR blank, lane 9 is JLS DNA, lane 10 is sterile sand, and lane 11 is background soil. FIG. 6B is a gel electrophoresis of the soil gene probe targeting the nidB-nidA region. Lane 1 is sterile sand, lane 2 is 100 bp DNA ladder plus, lane 3 is JLS, lane 4 is LTU soil extraction, lane 5 is LTU soil extraction, lane 6 is 100 bp DNA ladder plus, lane 7 is JLS DNA, lane 8 is PCR blank, lane 9 is sterile sand, and lane 10 is background soil. FIG. 6C is a gel electrophoresis of the soil gene probe targeting the nidA region. Lane 1 is sterile sand, lane 2 is 100 bp DNA ladder plus, lane 3 is JLS, lane 4 is LTU soil extraction, lane 5 is LTU soil extraction, lane 6 is 100 bp DNA ladder plus, lane 7 is blank, lane 8 is JLS DNA, lane 9 is sterile sand, and lane 10 is background soil.

The positive results in probe test are evident by the dominant bands that are approximately 510 bp for the nidB primer set, 471 bp for the nidB to nidA primer set and 735 bp for the nidA primer set. The dominant bands of DNA that were present in the gel probes were presumed to be amplified nid genes from native PAH-degrading mycobacteria in the soil. This presumption was verified by extracting and sequencing two of these bands. It is therefore a reasonable assumption to assume these three distinct PAH-degrading organisms may still survive in the soil. Other uncultured PAH-degrading mycobacteria may also survive in the soil.

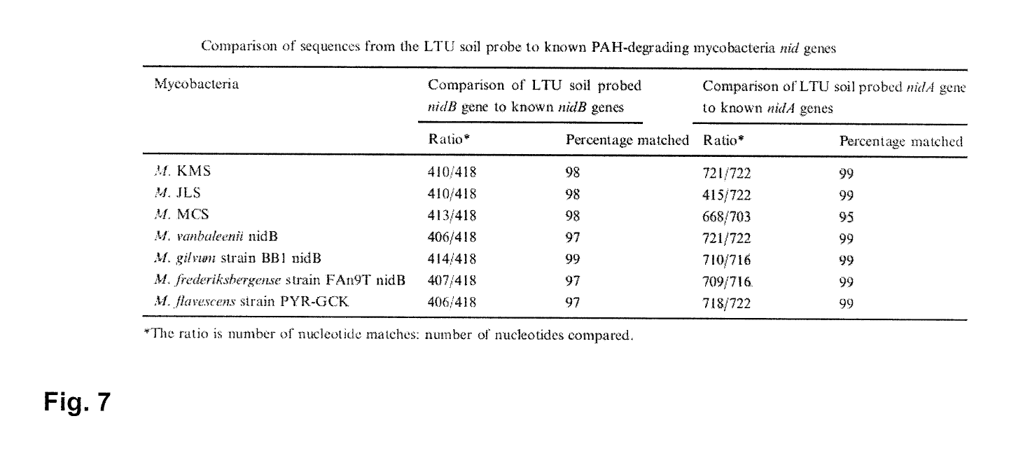

The sequencing yielded indeterminate regions notably after the first 21 nucleotides of the nidA gene there is a 9 bp insertion in MCS that is not present in the other nid genes. As suspected the nidA sequences from the LTU soil probe was indeterminate in this region. Although there were indeterminate regions, substantial regions of the nidB and nidA gene sequences were obtained, including a 420 bp region for nidB and a 722 bp region for nidA. These regions were compared to the nid gene sequences from seven known PAH-degrading mycobacteria. The results indicate a 97-98% identity match for the nidB gene and a 95 to 99% identity match for the nidA gene, as indicated in FIG. 7.

The identity matches indicate that the positive gene probe results by the electrophoreses gels of the PCR reactions do in fact represent detection of nid genes from PAH degrading mycobacteria in the LTU soil. The percentage of identity matches between the soil probe products and the known PAH-degrading mycobacteria nid genes is similar to the natural identity between known PAH-degrading mycobacteria nid genes within this soil system.

The results of this nid dioxygenase gene probe test indicate that this probe can be developed into a useful tool in bioremediation. The probe can be expanded for many uses including quantitative real time PCR and functional gene probe arrays. In order to use the probe for monitoring of bioremediation, the probe can be combined with quantitative PCR or real time PCR techniques. The PCR techniques employed in the current study involved conventional PCR methods. However, the template quantifying capabilities of real time PCR can be used with the same soil DNA extraction techniques and PCR primer sets used in this study to allow quantification of nid dioxygenase DNA in soil samples for monitoring of in situ soil bioremediation.

The genetic information and PCR primer sets can be an invaluable addition to the creation of functional gene probe arrays for the detection of soil bioremediation capabilities and for monitoring biodegradation activities. The nid dioxygenase probe can be combined with dioxygenase probes derived from the pseudomonad dioxygenases. This information on dioxygenase enzymes that are functionally specific to PAH metabolism can be combined with similar genes for catabolic enzymes involved in the transformations of environmental pollutants such as methyl tert-butyl ether, trichloroethylene, toluene and other common pollutants. By probing a soil for these catabolic genes, a functional gene probe array is performed that will be useful in classifying bioremediation capabilities of soils contaminated with mixed wastes. This array will expand the classification abilities of gene probes for detection of PAH biodegrading organisms in soils and other environmental matrices including sediments and biofilms. The probe array can be performed using multiple PCR reactions or using microarray technology.

The nid dioxygenase gene probes as described herein and in the sequence listing can be used to aid in detecting the presence of PAH degrading Mycobacteria in soils. Detection of these organisms gives information on the biodegradation and bioremediation capabilities of the indigenous microbial communities of contaminated soils. The nid probe was designed to be specific to PAH degrading mycobacteria. The probe was built as a soil probe by using a soil DNA extraction technique that was able to extract PCR quality DNA from mycobacteria in a soil matrix. The test results demonstrate that the nid dioxygenase soil gene probe is capable of identifying nid genes in soils undergoing bioremediation. The probe has been verified by sequencing the PCR products and comparing the sequences to known PAH-degrading mycobacteria nid genes. The test verification indicates a high degree of similarity (95-99%) between the probe and the nid dioxygenase genes of known PAH degrading mycobacteria.

### Example 10

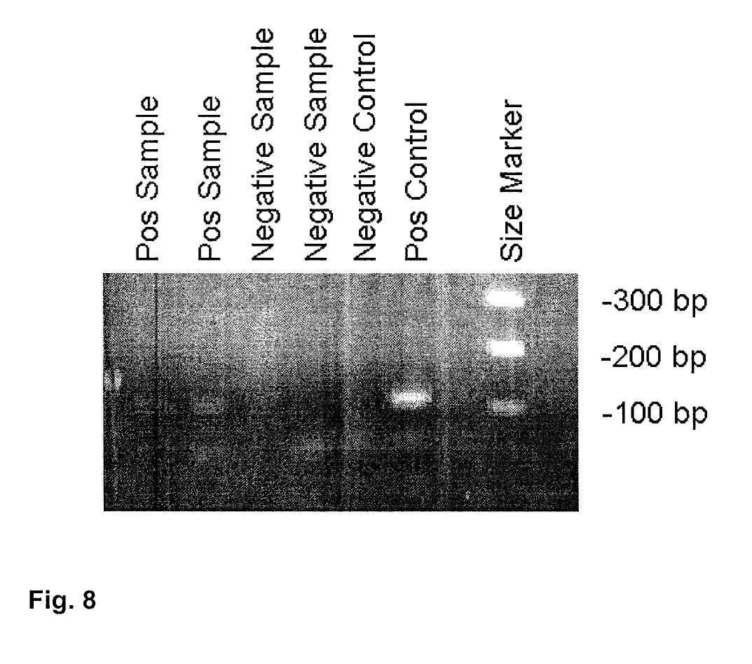

After the DNA was extracted from the PAH-contaminated soils, PCR products were prepared to determine if the DNA included the nid dioxygenase genes, which is a strong indication of the presence of PAH-biodegrading mycobacteria. The primers included: nidBpro-left (SEQ ID NO: 42): TCCATTCGGAGATGTTGTCTT; and nidBpro-right (SEQ ID NO: 43): CTCGACCAGACCATCAACACT. The primer nidBpro-left is obtained from the genetic DNA, and is obtained from a sequence located from −124 to −104 bases upstream (5′) to the nidB start ATG. Similarly, the primer nidBpro-right is located from −33 to −13 bases upstream (5′) to the nidB start ATG. FIG. 8 indicates that positive PCR reactions were obtained, and result in a DNA product size of 111 base pairs.

### Example 11

Experiments were conducted to determine substances that can be used in media to support growth and propagation of contaminant-degrading mycobacteria. As such, strains of M. KMS, JLS and MCS from the Libby sites and strains M. flavescens (“flav”) and M. vanbalenni (“PYR-1”) along with a standard M. smegmatis (“smeg”) were grown on media composed of various substances. Table 1 shows the substrates used by M. KMS, JLS and MCS from the Libby sites.

TABLE 1

| | | |
|---|---|---|
| KMS | MCS | JLS |

Common Substrates

| | | | |
|---|---|---|---|
| Tween 40 | x | x | x |
| Tween 80 | x | x | x |
| D-Fructose | x | x | x |
| D-Mannose | x | x | x |
| D-Trehalose | x | x | x |
| Pyruvic Acid Methyl Ester | x | x | x |

Differential Substrates

| | | | |
|---|---|---|---|
| D-Mannitol | x | | |
| D-Psicose | x | | |
| Propionic acid | | x | |
| D-Sorbitol | x | | |
| Sucrose | x | | |
| α-Cyclodextrin | | | x |
| Sedoheptulosan | x | | |

Table 1 shows that the tweens, mannose, fructose, trehalose, and pyruvic acid methyl ester were commonly used by the M. KMS, JLS and MCS strains and could be formulated to boost mycobacterium inocula over other bacteria. As such, these compounds can be used as carbon sources for growing and propagating the contaminant-degrading mycobacteria.

Table 2 shows the substrates used by M. KMS, M. flavescens (“flav”) and M. vanbalenni (“PYR-1”) along with a standard .M smegmatis (“smeg”) were grown on media composed of various substances.

TABLE 2

| | | | |
|---|---|---|---|
| KMS | Pyr-1 | flav | smeg |

Common Substrates

| | | | | |
|---|---|---|---|---|
| Tween 40 | x | x | x | x |
| Tween 80 | x | x | x | x |
| D-Fructose | x | x | x | x |
| D-Mannose | x | x | x | x |
| Sedoheptulosan | x | x | x | x |
| D-Sorbitol | x | x | x | x |

Novel Substrates

| | | |
|---|---|---|
| Dextrin | | x |
| L-Arabinose | | x |
| D-Arabitol | | x |
| D-Cellobiose | | x |
| D-Gluconic Acid | | x |
| α-D glucose | | x |
| 3-Methyl-D-Glucose | | x |
| α-Methyl-D-Glucoside | | x |
| Xylitol | | x |
| Acetic Acid | x | |
| α-Hydroxybutyric Acid | x | |
| β-Hydroxybutyric Acid | | x |
| Lactamide | | x |
| Succinic Acid | | |
| N-Acetyl-L-Glutamic Acid | x | |
| Glycerol | | x |

Based on Tables 1 and 2, the identification of carbon preferences by M. KMS, M. flavescens (“flav”) and M. vanbalenni (“PYR-1”) along with a standard M. smegmatis (“smeg”) can be used to select carbon sources that can be advantageously combined with a mycobacteria medium for M. KMS, JLS and MCS strains. The carbon sources can include D-mannitol, D-psicose, propionic acid, D-sorbitol, sucrose, alpha-cyclodextrin, and sedoheptulosan. More preferably, the carbon sources can include tween 40, tween 80, D-fructose, D-mannose, D-trehalose, and pyruvic acid methyl ester.

However, certain carbon sources have been identified to be unfavorable for use in a mycobacteria medium for M. KMS, JLS and MCS strains, or included in minimal quantities so as to prevent injury to the mycobacteria. As such, less favorable carbon sources that can be excluded from a mycobacteria medium can include dextrin, L-arabinose, D-arabitol, D-cellobiose, D-gluconic acid, alpha-D-glucose, alpha-methyl-D-glucose, xylitol, erythritol, acetic acid, alpha-hydroxybutric acid, beta-hydroxybutric acid, lactamide, succinic acid, N-acetyl-L-glutamic acid, and glycerol.

### Example 12

Studies were conducted to determine whether or not mycobacteria are capable of forming a biofilm in the presence of a root wash. Briefly, isolated M. KMS, JLS and MCS strains, M. flavescens (“flav”), and M. vanbalenni (“PYR-1”) were grown in the presence of Middlebrooks medium or a barley root wash. Liquid medium (e.g., root wash and commercial Middlebrooks) were inoculated and grown with shaking for 10 days. Initial inocula were 10⁵-10⁶ cfu/mL. The mycobacteria where then analyzed to determine the extend of biofilm formation.

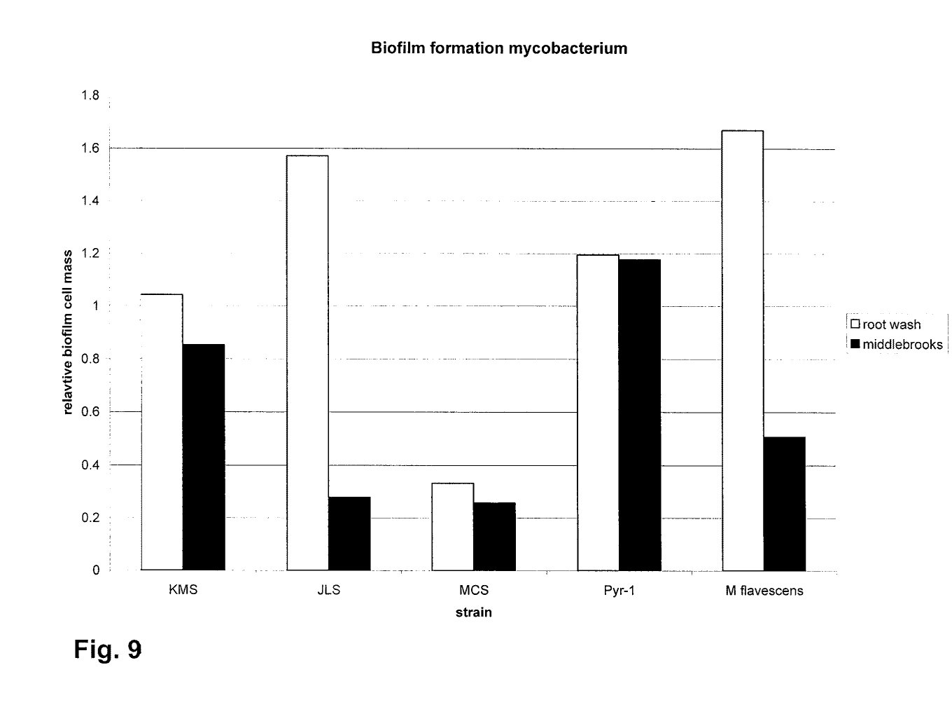

FIG. 9 shows that different mycobacterium strains have different potential for biofilm formation in the presence of root wash compared to Middlebrooks medium. Root wash permitted better biofilm formation compared to a complex commercial medium called Middlebrooks. More particularly, the isolated M. KMS, JLS and MCS strains showed enhanced biofilm formation in root wash compared to Middlebrooks medium. Thus, the substances within a root can be useful for growth and propagation of mycobacteria, especially for the isolated M. KMS, JLS and MCS strains.

### Example 13

Studies were conducted to determine whether or not mycobacteria are capable of planktonic growth in the presence of a root wash. Briefly, isolated M. KMS, JLS and MCS strains, M. flavescens (“flav”), and M. vanbalenni (“PYR-1”) were grown in the presence of Middlebrooks medium or a barley root wash. Liquid medium (e.g., root wash and commercial Middlebrooks) were inoculated and grown with shaking for 10 days. Initial inocula were 10⁵-10⁶ cfu/ml. The mycobacteria where then analyzed to determine the extent of planktonic growth.

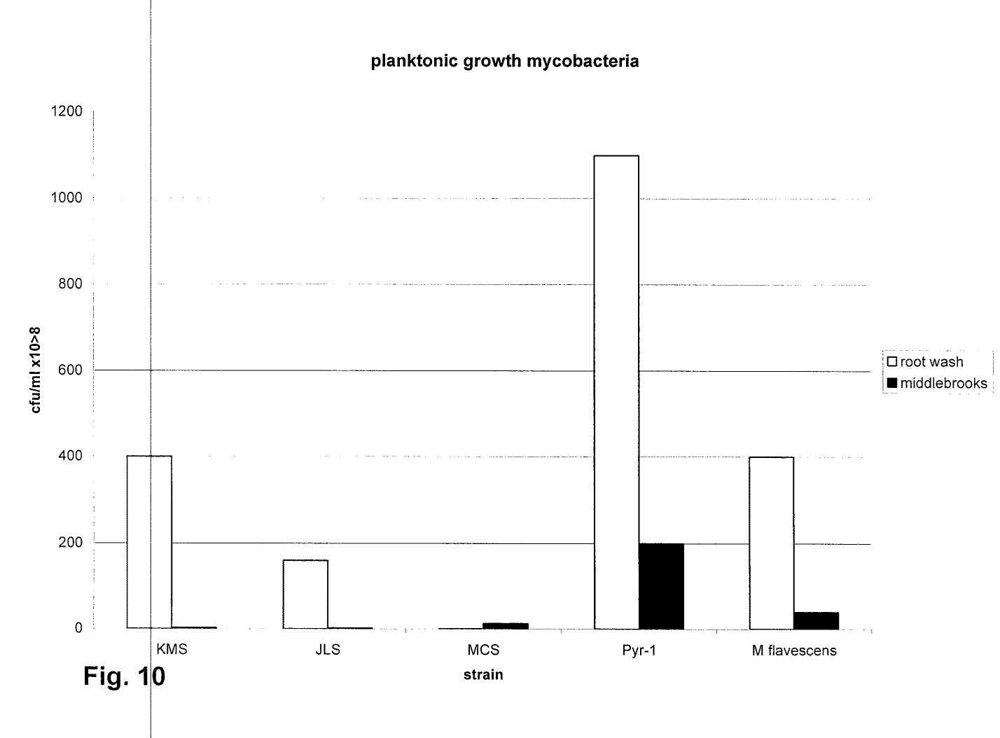

FIG. 10 shows that different mycobacterium strains have different potential for planktonic growth in the presence of root wash compared to Middlebrooks medium. The isolated M. KMS and JLS strains showed enhanced planktonic growth in root wash compared to Middlebrooks medium. On the other hand, MCS did not show enhanced planktonic growth. Thus, the substances within a root can be useful for growth and propagation of mycobacteria, especially for the isolated M. KMS, JLS and MCS strains. Also, there were differences in the final cell densities between the strains with PYR-1 being greater than KMS and M. flavescens.

### Example 14

It has been demonstrated that the mycobacteria M. KMS, JLS and MCS strains can colonize barley roots after seed inoculation and planting into sterile medium or soils containing natural microflora. Strong colonization is apparent in five day old inoculated seedlings.

### Example 15

Studies were conducted to determine whether or not mycobacteria can present strong colonization of plant roots in soils with native microbes present. Briefly, barley seeds were inoculated with the Libby M. KMS, by immersion into a suspension of 10⁹ colony forming units/ml, or were planted without inoculation. Seeds were planted at a depth of 1 inch into either background uncontaminated soil or PAH-contaminated soil from the Libby, MT site. After 14 days, roots were removed gently, vortexed in 5 mL sterile water for 1 minute and dilution plated onto Kings medium B agar plates with and without rifampicin and tetracycline to determine KMS and total bacterial colonies.

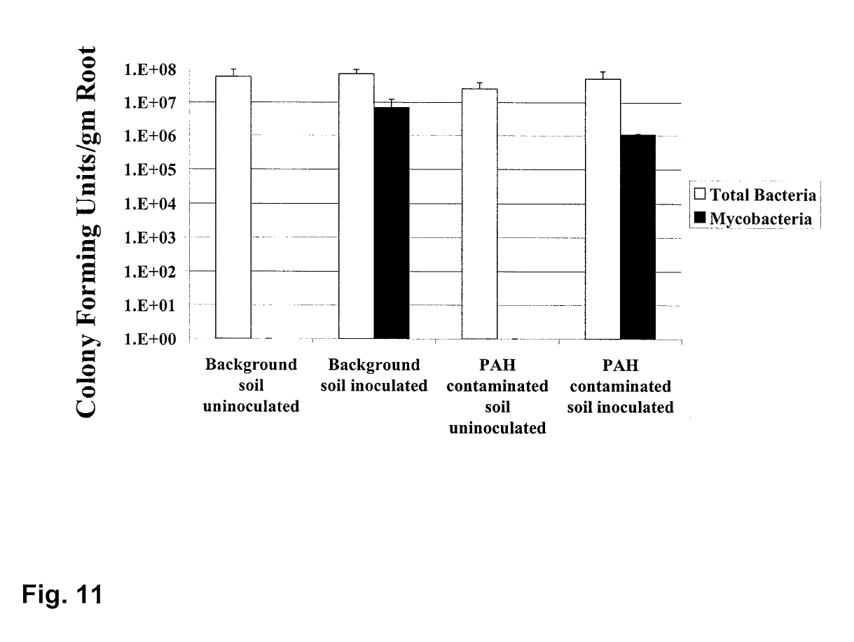

FIG. 11 shows the Libby mycobacterium isolate KMS colonized barley roots from a seed-borne inoculum. Similar findings are obtained with the other two Libby isolates. The mycobacterium was recovered from the roots at high levels in relation to total cell recovery after growth for ten days in a soil containing a normal microbial load (10⁷⁻⁸ cfu/g).

### Example 16

Seeds can be prepared prior to being coated with a mycobacteria composition. Briefly, seeds were processed to remove endogenous surface microbes and microbial endophytes. Seeds were immersed in 30% hydrogen peroxide for 5 minutes and washed with sterile water for three minutes, followed by three subsequent one-minute washes with sterile water to remove any remaining hydrogen peroxide. The seeds were heat-treated by suspension in sterile water at 50° C. for 30 minutes. After the heat treatment step, the seeds were surface-sterilized again following the hydrogen peroxide method previously described.

Treated seeds were plated on LB agar plates and incubated at 22° C. for up to 48 h to permit germination and detection of fungal or bacterial contamination. Seeds that showed signs of microbial contamination were discarded. Clean seeds were inoculated by submersing them in a suspension of mycobacterium cells for 30 seconds.

### Example 17

Seedlings can be grown in sterile environments. Briefly, the inoculated seeds of Example 7 were tested planted in sterile vermiculite for seedling growth. This growth matrix was prepared by adding 125 mL sterile water to approximately 325 mL vermiculite in Magenta boxes, and sterilizing at 121° C. for 40 minutes. After storing at room temperature for 24 h to allow fungal and bacterial spore germination, the boxes were sterilized again at 121° C. for 40 minutes. Three seeds were planted per container, and the plants were grown gnotobiotically at 26° C.

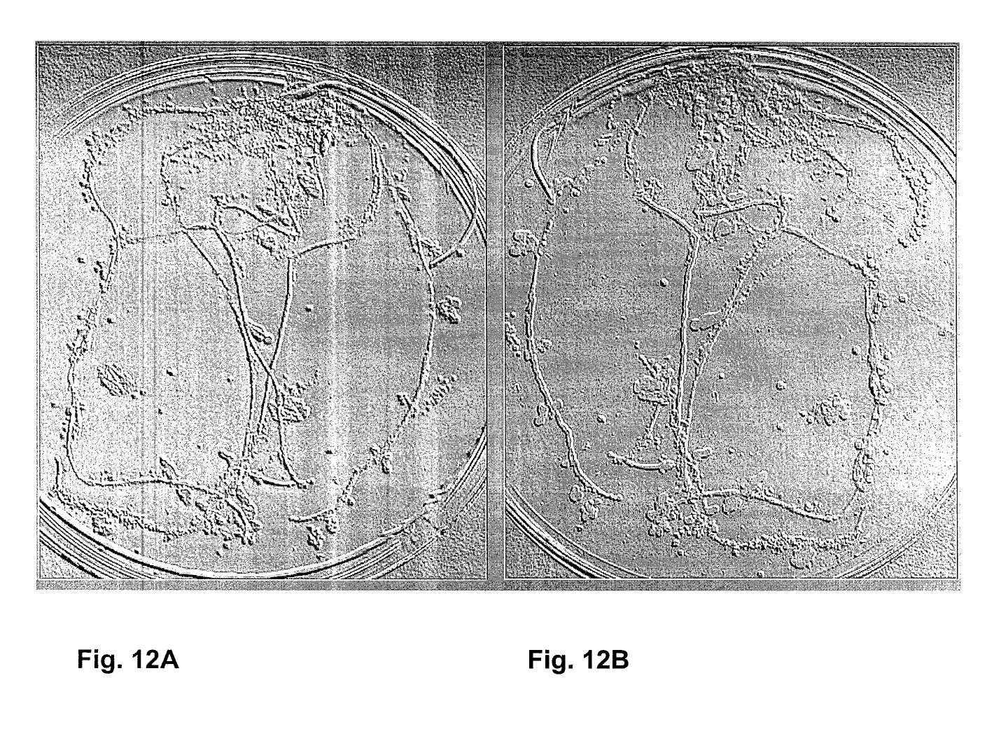

Strong colonization is apparent in 7 day old inoculated seedlings. This was determined by harvesting roots at 7 days, and blotted the root onto LB plate medium. The roots were incubated for 15 days before photographs were taken of the colonies. FIG. 12A is a top view of the colonies forming around the root, and FIG. 12B is a bottom view. PCR was used to confirm the identity of bacterial colonies as mycobacteria.

### Example 18

Barley seeds were prepared to include mycobacterium adhered to the outer surface of the seed. Briefly, cells were grown on amended Middlebrook 7H9 liquid medium. The cells were harvested during log-phase growth after five days, washed twice in sterile water, and suspended in sterile water. To determine the number of mycobacterium cells adhering to each seed, barley seeds inoculated as previously described were submersed in 1 mL sterile water and vortexed for 30 seconds. Serial dilutions of the water fractions were then performed and cfu/mL of cells were determined. The final cell density of the inoculum was approximately 10₈ cfu/mL.

### Example 19

Different sections of roots grown from barley seeds coated with mycobacteria were assayed to determine whether different sections of roots were better at sustaining mycobacteria growth. Briefly, roots that were not used in direct plating were harvested and dissected into 2 cm sections and vortexed in 1 mL sterile water for 30 seconds. Serial dilutions were made from the water onto LB plate medium and the number of mycobacterium colonies was determined for the different root sections. PCR was used to confirm the identity of the colonies. Serial dilutions from root sections of uninoculated sterile control seedlings were performed, and no microbial contamination was observed.

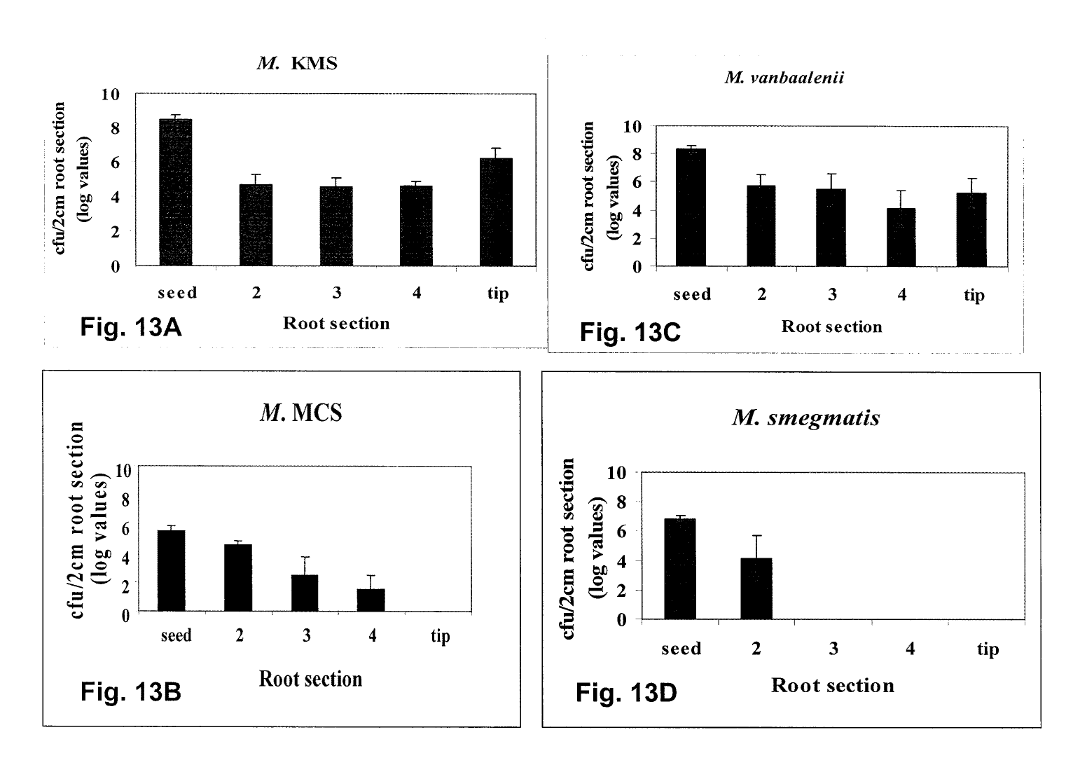

FIGS. 13A-13D show the colonization of mycobacteria on different root sections. Both KMS and M. vanbalenni show colonization of the root tip, which is a classic indication of strong colonizing mycobacteria. As such, FIGS. 13A-13D show that the root may serve as a type of bioinjector, and can be used to transport bacteria to different soil levels and pockets of contamination.

### Example 20

Studies were conducted to compare phytobioremediation against phytoremediation and bioremediation. As such, four conditions were tested as follows: sterile, uninoculated radiolabeled pyrene-amended sand (control); uninoculated barley only; M. KMS only; and barley inoculated with M. KMS. Each of the conditions was grown in a closed environment. Briefly, air was pumped through the system for 4 hours every 24 hour period, and 1 mL samples of a CO₂ trap solution were taken every two days and radioactivity counts were read using a scintillation counter. The experiment period was 10 days. After the experiment was terminated, ¹⁴C levels in the barley roots and leaves were determined by combustion and ¹⁴CO₂ collection. The ¹⁴C amounts in the sand were also determined by combustion.

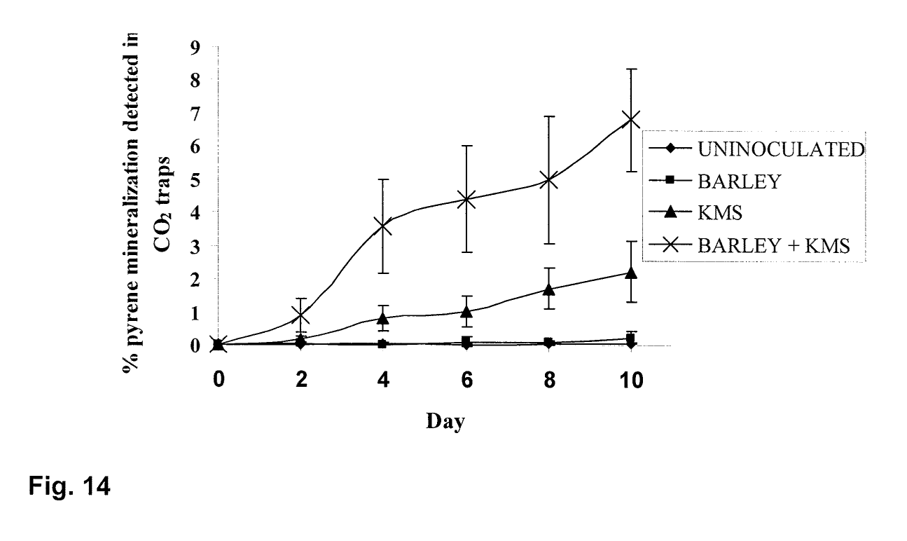

FIG. 14 shows that the phytobioremediation using barley and M. KMS was superior to phytoremediation with barley and bioremediation with M. KMS (The data shown in FIG. 14 are the mean of three independent experiments ± standard deviations). Also, bioremediation was superior to phytoremediation.

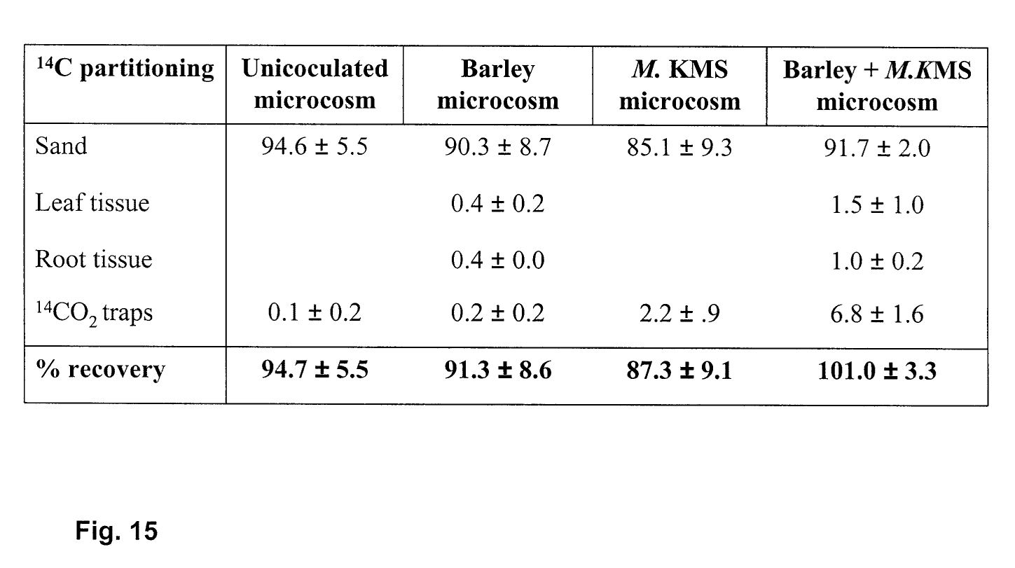

FIG. 15 is a mass balance of ¹⁴C. As such, the table shows that the ¹⁴C was preferentially relocated from the soil to the ¹⁴CO₂ collection traps compared to roots and leaves.

### Example 21

The mycobacteria M. KMS, JLS, and MCS strains were tested for the ability to mineralize MTBE and tertbutyl acetate (“TBA”), and compared against the mineralization ability of mycobacterium PM-1, and two bacteria cultured from a Ronan site. Microcosms were incubated statically in the dark at 32° C. for optimum temperature. Controls included one pure culture of each microbe was not spiked with MTBE or TBA. Concentrations of MTBE and TBA were set at 5 mg/L.

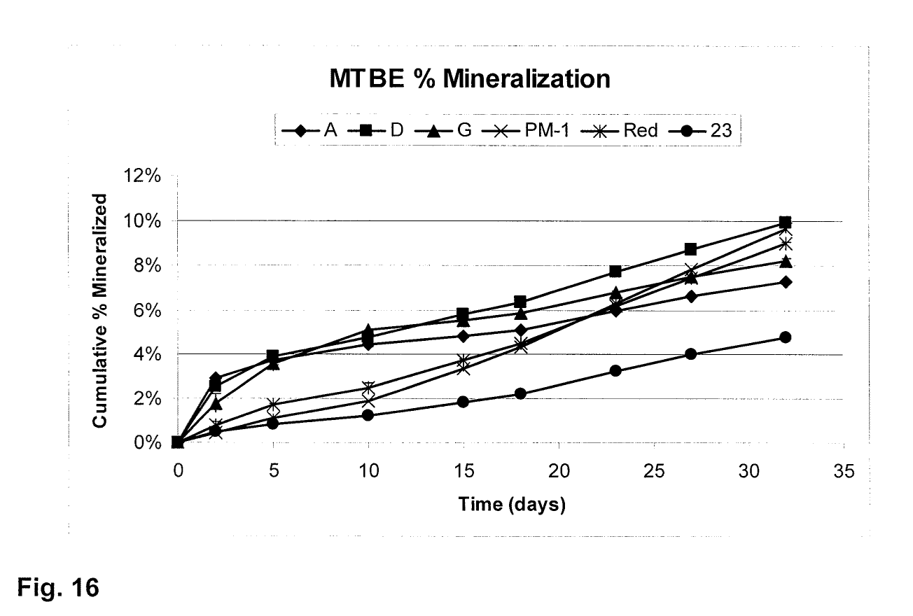

FIG. 16 is a graph of the ability of the M. KMS (shown as D), JLS (shown as A) and MCS (shown as G) strains to mineralize MTBE compared against mycobacterium PM-1 (pos control), and two bacteria cultured from a Ronan site (shown as Red and 23). As shown, M. KMS was superior in degrading MTBE.

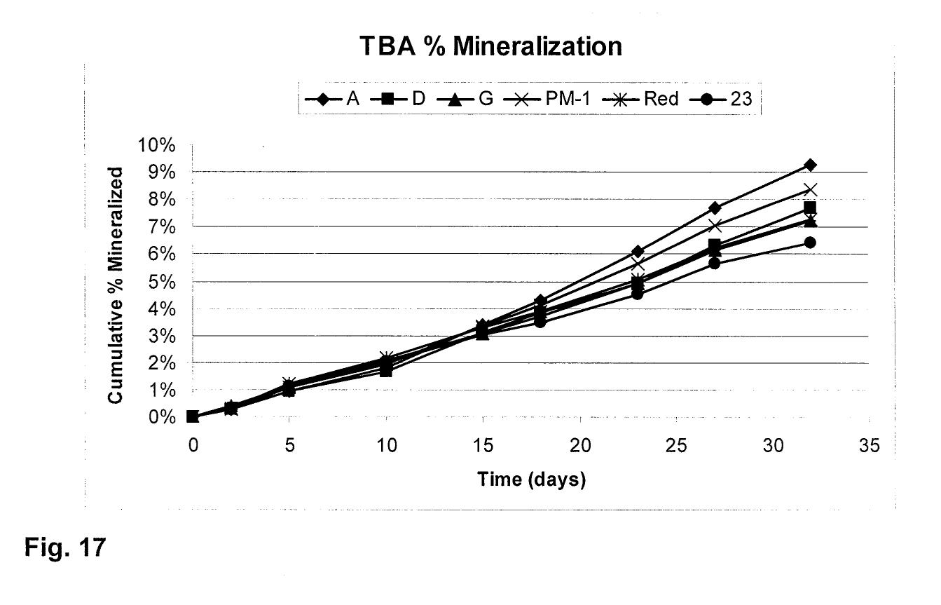

FIG. 17 is a graph of the ability of the M. KMS (shown as D), JLS (shown as A) and MCS (shown as G) strains to mineralize TBA compared against mycobacterium PM-1 (pos control), and two bacteria cultured from a Ronan site (shown as Red and 23). As shown, M. JLS was superior in degrading TBA.

### Example 22

The mycobacteria M. KMS, JLS, and MCS strains were tested for the ability to mineralize MTBE and tertbutyl acetate (“TBA”) in water, and compared against the mineralization ability of mycobacterium M. flavescens, and M. vanbalenni. Microcosms were incubated statically in the dark at 32° C. for optimum temperature. Controls included one pure culture not spiked with MTBE or TBA, and a culture with a distilled water spike. Concentrations of MTBE and TBA were set at 5 mg/L, and traps were sampled after 4 months.

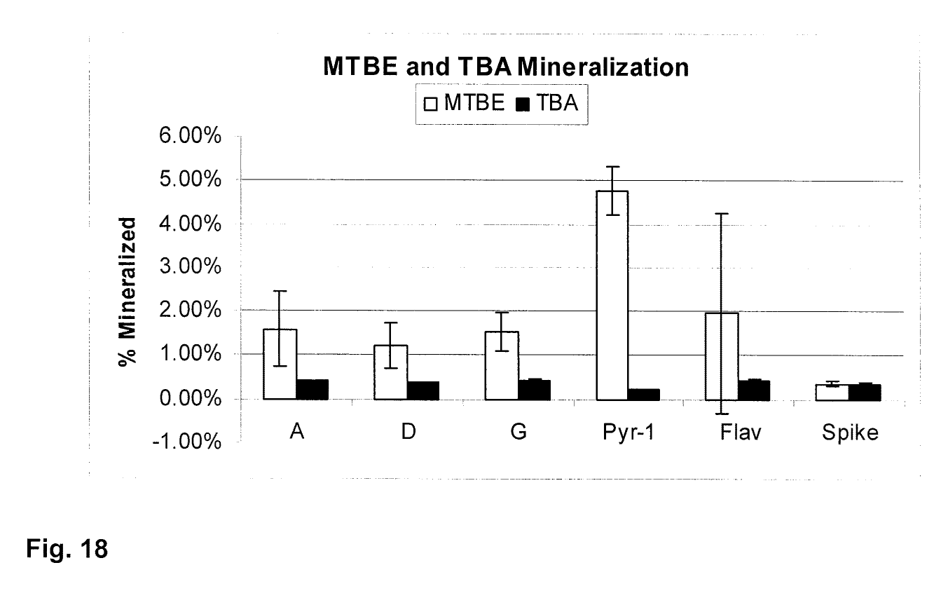

FIG. 18 is a graph of the ability of the M. KMS (shown as D), JLS (shown as A) and MCS (shown as G) strains to mineralize MTBE and TBA in water compared against M. flavescens (“flav”), and M. vanbalenni (“PYR-1”). As shown, all of the mycobacteria were capable of enhanced MTBE mineralization compared to TBA mineralization. Additionally, flav and PYR-1 had higher mineralization for MTBE compared to M. KMS, JLS, and MCS.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for determining whether a microorganism is capable of biodegrading a polycyclic aromatic hydrocarbon, the method comprising:
providing a first set of DNA molecules consisting of fragments of genomic DNA of at least one mycobacteria species capable of biodegrading a polycyclic aromatic hydrocarbon;
contacting, under hybridizing conditions, the first set of DNA molecules with a second set of DNA molecules consisting of genomic DNA of a microorganism, wherein it is not known whether or not the microorganism biodegrades a polycyclic aromatic hydrocarbon; and
detecting hybridization between the first set of DNA molecules and the second set of DNA molecules, wherein the hybridization between the first and second sets is an indication that the microorganism is capable of biodegrading a polycyclic aromatic hydrocarbon.

2. A method as in claim 1, wherein the detecting includes performing a polymerase chain reaction to amplify the amount of the second set of DNA molecules, and a first portion of the first set of DNA molecules includes a plurality of primers, wherein each of the primers is comprised of a primer nucleotide sequence having from about 8 to about 30 nucleic acids and hybridizes with at least one of the following:
a first nucleotide sequence which consists of SEQ ID NO: 1 or a complement of SEQ ID NO: 1;
a second nucleotide sequence which consists of SEQ ID NO: 3 or a complement of SEQ ID NO: 3;
a third nucleotide sequence which consists of SEQ ID NO: 5 or a complement of SEQ ID NO: 5;
a fourth nucleotide sequence which consists of SEQ ID NO: 7 or a complement of SEQ ID NO: 7;
a fifth nucleotide sequence which consists of SEQ ID NO: 9 or a complement of SEQ ID NO: 9; or
a sixth nucleotide sequence which consists of SEQ ID NO: 11 or a complement of SEQ ID NO: 11.

3. A method as in claim 2, wherein at least one primer sequence of the plurality of primers hybridizes with a conserved nucleotide sequence in each the following:
a first nucleotide sequence which consists of SEQ ID NO: 1;
a second nucleotide sequence which consists of SEQ ID NO: 3; and
a third nucleotide sequence which consists of SEQ ID NO: 5; or
a first nucleotide sequence which consists of a complement of SEQ ID NO: 1;
a second nucleotide sequence which consists of a complement of SEQ ID NO: 3; and
a third nucleotide sequence which consists of a complement of SEQ ID NO: 5.

4. A method as in claim 3, wherein at least one primer sequence of the plurality of primers hybridizes with a conserved nucleotide sequence in each the following:
a first nucleotide sequence which consists of SEQ ID NO: 7;
a second nucleotide sequence which consists of SEQ ID NO: 9; and
a third nucleotide sequence which consists of SEQ ID NO: 11; or
a first nucleotide sequence which consists of a complement of SEQ ID NO: 7;
a second nucleotide sequence which consists of a complement of SEQ ID NO: 9; and
a third nucleotide sequence which consists of a complement of SEQ ID NO: 11.

5. A method as in claim 4, wherein each primer is about 21 nucleic acids in length.

6. A method as in claim 5, wherein the primer nucleotide sequences are selected from the group consisting of:
SEQ ID NO: 26 or a complement of SEQ ID NO: 26;
SEQ ID NO: 27 or a complement of SEQ ID NO: 27;
SEQ ID NO: 28 or a complement of SEQ ID NO: 28;
SEQ ID NO: 29 or a complement of SEQ ID NO: 29;
SEQ ID NO: 30 or a complement of SEQ ID NO: 30;
SEQ ID NO: 31 or a complement of SEQ ID NO: 31;
SEQ ID NO: 32 or a complement of SEQ ID NO: 32;
SEQ ID NO: 33 or a complement of SEQ ID NO: 33.
SEQ ID NO: 34 or a complement of SEQ ID NO: 34;
SEQ ID NO: 35 or a complement of SEQ ID NO: 35; and combinations thereof.

7. A method as in claim 5, wherein the primer nucleotide sequences are not members of the group consisting of:
SEQ ID NO: 26 or a complement of SEQ ID NO: 26;
SEQ ID NO: 27 or a complement of SEQ ID NO: 27;
SEQ ID NO: 28 or a complement of SEQ ID NO: 28;
SEQ ID NO: 29 or a complement of SEQ ID NO: 29;
SEQ ID NO: 30 or a complement of SEQ ID NO: 30;
SEQ ID NO: 31 or a complement of SEQ ID NO: 31;
SEQ ID NO: 32 or a complement of SEQ ID NO: 32; and
SEQ ID NO: 33 or a complement of SEQ ID NO: 33.

8. A method as in claim 4, wherein a second portion of the first set of DNA molecules includes a second plurality of primers, wherein each of the primers in the second plurality is comprised of a primer nucleotide sequence having from about 8 to about 30 nucleic acids and hybridizes with a conserved nucleotide sequence within each of the following:
a seventh nucleotide sequence which consists of SEQ ID NO: 13;
a eighth nucleotide sequence which consists of SEQ ID NO: 14;
a ninth nucleotide sequence which consists of SEQ ID NO: 15;
a tenth nucleotide sequence which consists of SEQ ID NO: 16; and
a eleventh nucleotide sequence which consists of SEQ ID NO: 17; or
a seventh nucleotide sequence which consists of a complement of SEQ ID NO: 13;
a eighth nucleotide sequence which consists of a complement of SEQ ID NO: 14;
a ninth nucleotide sequence which consists of a complement of SEQ ID NO: 15;
a tenth nucleotide sequence which consists of a complement of SEQ ID NO: 16; and
a eleventh nucleotide sequence which consists of a complement of SEQ ID NO: 17.

9. A method as in claim 8, wherein a third portion of the first set of DNA molecules includes a third plurality of primers, wherein each of the primers in the third plurality is comprised of a primer nucleotide sequence having from about 8 to about 30 nucleic acids and hybridizes with a conserved nucleotide sequence within each of the following:
a twelfth nucleotide sequence which consists of SEQ ID NO: 18;
a thirteenth nucleotide sequence which consists of SEQ ID NO: 19;
a fourteenth nucleotide sequence which consists of SEQ ID NO: 20;
a fifteenth nucleotide sequence which consists of SEQ ID NO: 21; and
a sixteenth nucleotide sequence which consists of SEQ ID NO: 22; or
a twelfth nucleotide sequence which consists of SEQ ID NO: 18;
a thirteenth nucleotide sequence which consists of SEQ ID NO: 19
a fourteenth nucleotide sequence which consists of SEQ ID NO: 20;
a fifteenth nucleotide sequence which consists of SEQ ID NO: 21; and
a sixteenth nucleotide sequence which consists of SEQ ID NO: 22.

10. A method as in claim 8, wherein the primers in the second plurality and third plurality are about 19 to about 22 nucleic acids in length.

11. A method as in claim 1, wherein the detecting includes performing a polymerase chain reaction to amplify the amount of the second set of DNA molecules, and a primer portion of the first set of DNA molecules includes a plurality of primers, wherein each of the primers is comprised of a primer nucleotide sequence consisting of at least one of the following:
a first nucleotide sequence which consists of SEQ ID NO: 42 or a complement of SEQ ID NO: 42; or
a second nucleotide sequence which consists of SEQ ID NO: 43 or a complement of SEQ ID NO: 43.

12. A method of identifying the presence of polycyclic aromatic hydrocarbon-degrading mycobacteria having nidB-nidA dioxygenase genes in a sample, the method comprising:
providing at least one primer capable of hybridizing with a nidB-nidA dioxygenase genomic DNA nucleotide sequence of at least one known polycyclic aromatic hydrocarbon-degrading mycobacterium;
contacting the at least one primer with a sample, wherein it is not known whether or not the sample includes a polycyclic aromatic hydrocarbon-degrading mycobacterium;
producing a polymerase chain reaction product; and
determining whether the polymerase chain reaction product indicates the presence of genomic DNA of a microorganism having a nidB-nidA dioxygenase nucleotide sequence in the sample.

13. A method as in claim 12, wherein the at least one primer is comprised of a primer nucleotide sequence having from about 19 to about 25 nucleic acids and hybridizes with at least three of the following:
a first nucleotide sequence which consists of SEQ ID NO: 1
a second nucleotide sequence which consists of a complement of SEQ ID NO: 1;
a third nucleotide sequence which consists of SEQ ID NO: 3;
a fourth nucleotide sequence which consists of a complement of SEQ ID NO: 3;
a fifth nucleotide sequence which consists of SEQ ID NO: 5;
a sixth nucleotide sequence which consists of a complement of SEQ ID NO: 5;
a seventh nucleotide sequence which consists of SEQ ID NO: 7
an eighth nucleotide sequence which consists of a complement of SEQ ID NO: 7;
a ninth nucleotide sequence which consists of SEQ ID NO: 9;
a tenth nucleotide sequence which consists of a complement of SEQ ID NO: 9;
an eleventh nucleotide sequence which consists of SEQ ID NO: 11; or
a twelfth nucleotide sequence which consists of a complement of SEQ ID NO: 11.

14. A method as in claim 13, wherein the at least one primer nucleotide sequence is about 20 to about 22 nucleic acids long.

15. A method as in claim 14, wherein the at least one primer nucleotide sequence is selected from the group consisting of:
SEQ ID NO: 26 or a complement of SEQ ID NO: 26;
SEQ ID NO: 27 or a complement of SEQ ID NO: 27;
SEQ ID NO: 28 or a complement of SEQ ID NO: 28;
SEQ ID NO: 29 or a complement of SEQ ID NO: 29;
SEQ ID NO: 30 or a complement of SEQ ID NO: 30;
SEQ ID NO: 31 or a complement of SEQ ID NO: 31;
SEQ ID NO: 32 or a complement of SEQ ID NO: 32;
SEQ ID NO: 33 or a complement of SEQ ID NO: 33.
SEQ ID NO: 34 or a complement of SEQ ID NO: 34; and
SEQ ID NO: 35 or a complement of SEQ ID NO: 35.

16. A method as in claim 13, further comprising:
collecting soil;
extracting genomic DNA from microorganisms in the soil; and
purifying the genomic DNA to obtain the sample.

17. A method as in claim 16, wherein the soil sample is from a site contaminated with polycyclic aromatic hydrocarbons, and the method further comprises at least one of the following:
freezing and thawing the microorganisms;
bead-beating the microorganisms; and
removing polymerase chain reaction inhibitors with binding resins.

18. A method as in claim 13, wherein the determining includes at least one of the following:
determining the size of the polymerase chain reaction product by electrophoresis with a DNA ladder;
determining the size of the polymerase chain reaction product by electrophoresis with a known nidB DNA, nidA DNA, and/or combinations thereof;
determining the size of the polymerase chain reaction product by electrophoresis with a known nidB DNA and/or nidA DNA from at least one of mycobacterium JLS, mycobacterium KMS, or mycobacterium MCS;
sequencing the polymerase chain reaction product to determine the nucleotide sequence thereof,
comparing the polymerase chain reaction product nucleotide sequence with a known polycyclic aromatic hydrocarbon-degrading mycobacterium nidA and/or nidB nucleotide sequence;
comparing the polymerase chain reaction product nucleotide sequence with a nidA and/or nidB nucleotide sequence from a known polycyclic aromatic hydrocarbon-degrading mycobacterium selected from the group consisting of mycobacterium JLS, mycobacterium KMS, mycobacterium MCS, mycobacterium vanbaalenii, mycobacterium frederiksbergense strain FAn9T, mycobacterium flavescens strain PYR-GCK;
comparing the polymerase chain reaction product nucleotide sequence with a known polycyclic aromatic hydrocarbon-degrading mycobacterium nidA and/or nidB nucleotide sequence, wherein a sequence nucleotide identity match greater than 95% indicates the sample contains a polycyclic aromatic hydrocarbon-degrading mycobacterium; or
comparing the polymerase chain reaction product nucleotide sequence with a known polycyclic aromatic hydrocarbon-degrading mycobacterium nidA and/or nidB nucleotide sequence, wherein a sequence nucleotide identity match greater than 97% indicates the sample contains a polycyclic aromatic hydrocarbon-degrading mycobacterium.

19. A method as in claim 13, further comprising:
providing at least one primer capable of hybridizing with a mycobacterium 16S ribosomal DNA nucleotide sequence;
hybridizing the at least one primer with the 16S ribosomal DNA nucleotide sequence; and
producing a polymerase chain reaction product; and
determining whether the polymerase chain reaction product indicates the presence of a polycyclic aromatic hydrocarbon-degrading mycobacterium.

20. A method as in claim 13, wherein the 16S ribosomal DNA nucleotide sequence is a promoter sequence.

21. A method as in claim 12, wherein the at least one primer is comprised of a primer nucleotide sequence consisting of at least one of the following:
a first nucleotide sequence which consists of SEQ ID NO: 42 or a complement of SEQ ID NO: 42; or
a second nucleotide sequence which consists of SEQ ID NO: 43 or a complement of SEQ ID NO: 43.
